SEQUENCE LISTING (10 sequences)

SEQ ID NO: 1 - DNA, 21 bp, synthetic construct
  source 1..21: mol_type = other DNA; note = Glyceraldehyde-3-phosphate dehydrogenase (GADPH) forward primer sequence
ttgccatcaatgaccccttca

SEQ ID NO: 2 - DNA, 19 bp, synthetic construct
  source 1..19: mol_type = other DNA; note = Glyceraldehyde-3-phosphate dehydrogenase (GADPH) reverse primer sequence
cgccccacttgattttgga

SEQ ID NO: 3 - DNA, 18 bp, synthetic construct
  source 1..18: mol_type = other DNA; note = Collagen alpha-1(I) chain (COL1A1) forward primer sequence
aggtccccctggaaagaa

SEQ ID NO: 4 - DNA, 18 bp, synthetic construct
  source 1..18: mol_type = other DNA; note = Collagen alpha-1(I) chain (COL1A1) reverse primer sequence
aatcctcgagcaccctga

SEQ ID NO: 5 - DNA, 21 bp, synthetic construct
  source 1..21: mol_type = other DNA; note = Runt-related transcription factor 2 (RUNX2) forward primer sequence
aaatgcctccgctgttatgaa

SEQ ID NO: 6 - DNA, 18 bp, synthetic construct
  source 1..18: mol_type = other DNA; note = Runt-related transcription factor 2 (RUNX2) reverse primer sequence
gctccggcccacaaatct

SEQ ID NO: 7 - DNA, 21 bp, synthetic construct
  source 1..21: mol_type = other DNA; note = Alkaline phosphatase, tissue-nonspecific isozyme (ALPL) forward primer sequence
atctttggtctggctcccatg

SEQ ID NO: 8 - DNA, 19 bp, synthetic construct
  source 1..19: mol_type = other DNA; note = Alkaline phosphatase, tissue-nonspecific isozyme (ALPL) reverse primer sequence
tttcccgttcaccgtccac

SEQ ID NO: 9 - DNA, 20 bp, synthetic construct
  source 1..20: mol_type = other DNA; note = Osteopontin (SPP1) forward primer sequence
agctggatgaccagagtgct

SEQ ID NO: 10 - DNA, 20 bp, synthetic construct
  source 1..20: mol_type = other DNA; note = Osteopontin (SPP1) reverse primer sequence
tgaaattcatggctgtggaa

(19) United States
(12) Patent Application Publication
(10) Pub. No.: US 2026/0069746 A1
(43) Pub. Date: Mar. 12, 2026

(54) SYNTHETIC BONE GRAFTS AND METHODS FOR THEIR PREPARATION

(71) Applicants: Mimetis Biomaterials, Barcelona (ES); Universitat Politècnica de Catalunya, Barcelona (ES)

(72) Inventors: Linh Ha Huong Lovisa Johansson, Barcelona (ES); Santiago Raymond Llorens, Barcelona (ES); Maria Pau Ginebra Molins, Barcelona (ES)

(21) Appl. No.: 19/107,305

(22) PCT Filed: Aug. 29, 2023
(86) PCT No.: PCT/EP2023/073679

§ 371 Date: Feb. 27, 2025

(30) Foreign Application Priority Data
Aug. 31, 2022 (EP) 22193226.2

(51) Int. Cl. A61L 27/46; A61L 27/56; B33Y 70/10; C09D 11/107

(52) U.S. Cl. CPC A61L 27/46; A61L 27/56; C09D 11/107; A61L 2400/12; A61L 2430/02; B33Y 70/10

(57) ABSTRACT

The present invention provides methods for the preparation of synthetic bone grafts which are made of a composition comprising two matrixes, one ceramic including interlocked CDHA crystals, and another of one or more binder(s), the two matrixes being admixture. The method comprises the preparing of an ink composition, the 3D-printing, and the hardening of the binder and ceramic components, in this order.

The resulting bone grafts, which are characterized by including the two matrixes in admixture, shows improved mechanical properties as well as excellent biological properties.

### CROSS REFERENCE TO RELATED APPLICATION

This application is a 371 National Stage of International Patent Application No. PCT/EP2023/073679, filed on Aug. 29, 2023, which claims priority to European Patent Application No. EP 22193226.2, filed Aug. 31, 2022, the disclosures of which are incorporated by reference herein in their entireties.

### TECHNICAL FIELD

The present invention relates to the field of bone graft substitutes. In particular, the invention provides 3D-printed synthetic bone grafts, as well as methods for their production.

### BACKGROUND ART

The recent developments in three dimensional (3D) printing technologies have opened vast opportunities for the development of patient-specific bone graft substitutes, which is expected to have a particularly high impact on dental and maxillofacial surgery as well as in trauma and orthopaedic surgery. Whereas autologous or allogenic bone grafts are still the gold standard in the frequent bone grafting surgical procedures performed nowadays, the possibility to fabricate customised scaffolds based on digitalised medical imaging techniques has provided a real boost to the applications of synthetic bone grafts. The novel fabrication approaches referred to as 3D printing techniques are based on computer-aided design (CAD) and computer-aided manufacturing (CAM) and result in a patient-specific bone grafting therapy. Such technologies allow the design of personalised synthetic bone grafts, matching the specificity of every defect in terms of both the metabolic and anatomic characteristics, opening new perspectives in this field.

The use of direct in writing (DIW) with calcium phosphate (CaP)-based inks for the fabrication of bone implants and scaffolds for bone tissue engineering was proposed for the first time in 2005. Several works have reported the fabrication and properties of robocasted scaffolds made of hydroxyapatite (HA), beta-tricalcium phosphate (P-TCP), biphasic combinations of 3-TCP and HA and bioactive glasses, for instance.

In spite of the efforts made, however, there remains several limitations to be overcome.

In some approaches, ceramic particles remain embedded in a binder matrix and can only provide a role as a filler.

In other approaches, based on the sintering process, the incorporation of the polymeric phase is limited to post-treatment steps, in the form of coatings, thus resulting in a composite having two separate phases. Furthermore, it requires high sintering temperatures, promoting the decomposition of the polymeric phase. This also gives rise to shrinkage and brittleness, together with a reduced biological performance compared to non-sintered ceramics.

When natural based materials were used (e.g. collagen, alginate, chitosan, gelatin), the major drawbacks found were their poor reproducibility, the difficulty in controlling the degradation rate and mechanical properties, the risk for disease transmission and their limited availability. Therefore, there is still the need of providing appropriate bone grafts overcoming one or more of the above-identified drawbacks.

### SUMMARY OF THE INVENTION

The present inventors have developed a novel composite scaffold design based on two intertwined matrixes, one ceramic and the other comprising the binder(s).

Up to now, composite scaffolds were made in particulate form, providing a limited exposition of the bioactive ceramic particles to the tissue (FIG. 1(a)). The present inventors, on the contrary, have designed a scaffold which, contrary to the state of the art, includes a continuous ceramic phase (matrix) based on interlocked CDHA crystals (see FIG. 1(b)).

In addition to the above, the continuous ceramic phase is in admixture with a continuous polymeric phase. This continuous polymeric phase renders the scaffold more though (mimicking the mechanical function of the collagen fibrils in native bone), makes the scaffold more biocompatible than sintered scaffolds, and shows enhanced fracture toughness compared to a pure ceramic scaffold hardened at low temperatures (see Examples below).

As the ceramic mineral phase, having a composition and microstructure close to the mineral phase of bone, entangles with the polymeric biocompatible phase, the scaffold is more similar to the native bone compared to other synthetic scaffolds.

The disposition of the ceramic and the binder in two continuous phases (matrixes), which are in admixture, provides significant remarkable improved properties to the bone grafts. In this regard, the inventors compared the behavior of some bone grafts of the invention with one already in the market under the name MimetikOss® 3D, which is a pure ceramic scaffold. As it is shown below, the inventors found that with the configuration of the ceramic particles and binder(s) in two intertwined matrixes, the flexural toughness was at least 3-fold increased and, at the same time, the compressive strength was increased in at least 3-fold.

The inventors confirmed that these improved mechanical properties translated into an enhanced fixability (see Example 11 below). The bone graft of the invention also shows excellent biological properties in vitro (Table 12 and Table 13 below).

In order to obtain the scaffolds with such innovative design, the inventors have developed a method which is based on performing, after the 3D-printing of the scaffold, two hardening steps, which have to be performed in the following order: firstly the hardening of the binder to obtain a polymeric matrix and, once formed, the hardening of the ceramic particles is subsequently performed to achieve a ceramic matrix with interlocked CDHA crystals.

Thus, in a first aspect the present invention provides a method for producing a synthetic bone graft comprising:
(a) Preparing an ink composition comprising a-TCP and one or more binders, this step comprising:
   a.1. preparing a binder solution comprising one or more non-water-soluble binders; or, alternatively, one or more water-soluble photo-crosslinkable binder(s), and
   a.2. adding α-TCP to the binder solution, this step (a) further comprising, when the ink composition comprises one or more photo-crosslinkable binder(s), the adding of one or more photoinitiator(s);
(b) 3D-printing the synthetic bone graft; and
(c) Subjecting the synthetic bone graft to conditions providing cohesion between the binder and the α-TCP particles, this step comprising:
   c.1. reducing the solvent content of the bone graft; or, alternatively,
   c.2. crosslinking the one or more photo-crosslinkable binder(s) in the presence of the photoinitiator.

In a second aspect the present invention provides a 3D-printed bone graft made of a composition comprising a ceramic matrix which is in admixture with a binder matrix, wherein:
the ceramic matrix comprises a crystalline phase including interlocked calcium-deficient hydroxyapatite (CDHA) crystals; and
the binder matrix is made from one or more non-water-soluble binders; one or more water-soluble photo-crosslinkable binders; or any mixture thereof;
the ceramic matrix is at a weight percentage of at least 50 wt % with respect to the total weight of the composition, and
the binder matrix is at a weight percentage in the range from 5 to 40 wt % with respect to the total weight of the composition.

In a third aspect the present invention provides a 3D-printed bone graft made of a composition comprising α-TCP particles in admixture with a binder matrix, the binder matrix being made from one or more non-water-soluble binders; from one or more water-soluble photo-crosslinked binders; or any mixture thereof; and wherein the α-TCP particles are at a weight percentage of least 50 wt % with respect to the total weight of the composition, and the binder matrix is at a weight percentage in the range from 5 to 30 wt % with respect to the total weight of the composition.

### BRIEF DESCRIPTION OF FIGURES

FIG. 1: schematic representation of the morphology of a 3D-printed strand composed of a polymeric phase and a ceramic phase. (a) ceramic particles are dispersed and embedded within a polymeric matrix, serving as fillers (comparative); (b) Ceramic phase with entangled nanocrystals which form an interconnected consolidated ceramic matrix, intertwined with the polymeric fibrils (invention).

FIG. 2: (a)-(f) is a sequence from a video taken with a digital camera (16:9 FDH 1920×1080, Samsung Galaxy S) demonstrating the flexibility of a 3D-printed scaffold before hardening of the ceramic phase, which may consolidate into a rigid scaffold in situ once implanted in the body and in contact with the body fluid.

FIG. 3: SEM images of the microstructure of 3D-printed scaffold (Example 3.4). Acquisition by BSE detector run at 15 kV (Phenom XL Desktop SEM, PhenomWorld), images from left to right, (a)-(d), taken at augmentations ×300, ×500, ×10 000 and ×19 000, respectively.

FIG. 4: SEM images of the microstructure in a crack of 3D-printed scaffolds: (a) MimetikOss® 3D (i.e., a pure ceramic scaffold as describes in the patent EP3563881A1 “Synthetic bone graft” following Example 5. Patient-specific defect), (b) scaffolds with PCL in the binder solution. Acquisition by BSE detector run at 15 kV (Phenom XL Desktop SEM, PhenomWorld), images taken at augmentation ×5000.

FIG. 5: X-ray powder diffraction spectra of 3D-printed scaffolds containing different amount of PLGA (Examples 3.10 and 3.13), and compared to MimetikOss® 3D (i.e., a pure ceramic scaffold as describes in the patent EP3563881A1 “Synthetic bone graft” following Example 5. Patient-specific defect). The crystalline phases were identified and quantified by intensity ratio method (DIFFRAC plusBASIC Evaluation Package, EVA, Bruker-AXS 2007). Samples were printed with a 25 Ga nozzle. The (---)line represents the CDHA crystals, the continuous line represents the α-TCP and the (⋅⋅⋅⋅) represents the β-TCP. I=intensity.

FIG. 6: Fourier-transform infrared (FTIR) spectra from 3D-printed scaffolds containing different amount of PLGA (Examples 3.10 and 3.13, named T18 and T65, respectively, in graph), and compared to MimetikOss® 3D (i.e., a pure ceramic scaffold as describes in the patent EP3563881A1 “Synthetic bone graft” following Example 5. Patient-specific defect) and pure PLGA (with L-lactide:Glycolide molar ratio of 65:35 and 82:18). Samples were printed with a 25 Ga nozzle. T=transmittance; \tilde{V}=wavenumber.

FIG. 7: represents the pore entrance size distribution analyzed in the range between 0.006 and 360 μm by mercury intrusion porosimetry (MIP) on 3D-printed scaffolds containing different amount of PLGA (Examples 3.10 (- - - -) and 3.13(⋅⋅⋅⋅⋅⋅), and compared to MimetikOss® 3D (continuous line). Samples were printed with a 25 Ga nozzle. Log d.i.=Lod differential intrusion; P.S.=pore size.

FIG. 8. Scanning electron microscopy (SEM) images taken from the scaffold cross-section (scale bar 200 μm), filament surface (scale bar 1 μm) and filament cross-section (scale bar 1 μm), showing the microstructure of the respective three conditions: bioceramic scaffolds (CTRL), composite scaffolds with PLGA in the ink (embodiment of the invention, PLGA-I) and PLGA as a coating (comparative purpose, PLGA-C). White arrows indicate parts of the polymeric phase.

FIG. 9. Screwability tests on CTRL, PLGA-1 and PLGA-C scaffolds in a design to reconstruct a challenging vertical and horizontal knife-edge ridge indication in the jaw, including the surgical steps: perforation of the scaffold and anatomical biomodel with a ø1.2 mm drill and fixation of the scaffold with a ø1.5 mm/L 7 mm dental screw. The asterisk (*) in the PLGA-I recovered sample: several perforated holes were drilled in the recovered scaffold post-fixation to assess the resistance to adjacent drilled holes.

FIG. 10. In vitro biological assessment employing hMSC cells incubated for 1, 7 and 21 days in direct contact with the different 3D-printed CaP-based scaffolds: bioceramic (CTRL), PLGA in the ink (embodiment of the invention, PLGA-I) and PLGA as a coating (comparative purpose, PLGA-C): (A) Cell viability evaluated with Presto Blue®; results are normalised relative to CTRL samples at each respective time-point; (B) Representative images of cell morphology and attachment of cells on the 3D-printed filaments (scale bar 50 μm); (C) Representative images of cell proliferation and cytoskeleton spreading of cells directly attached to the 3D-printed filaments (scale bar 500 μm), white arrows indicates interconnected cytoskeletons, bridging between cells; (D) Representative images of the scaffold cross-section after 21 days of cell culture (scale bar 50 μm), white arrows indicate cells bridging the macropores between filaments in PLGA-I and PLGA-C. All images in (B)-(D) were acquired by fluorescent CLSM, F-Actin (orange) and nuclei (blue); (E) ALPL, RUNX2, SPP1 and COL1A1 gene expression of hMSC cultured on scaffolds for 21 days; expression levels were determined by qRT-PCR and results were normalised relative to CTRL samples, using GAPDH as housekeeping gene; (F) Alkaline phosphatase (ALP) activity quantified by absorbance measurements. In (A), (E) and (F) different letters and numbers indicate statistically significant differences (p<0.05) between conditions within each time-point and gene.

FIG. 11 represents in vitro biological assessment with hMSC cells incubated for 1 day in direct contact with the different 3D-printed CaP-based scaffolds: bioceramic (CTRL), PLGA in the ink (embodiment of the invention, PLGA-I) and PLGA as a coating (comparative purpose, PLGA-C). Representative images of cell morphology and attachment to the 3D-printed filaments, acquired by fluorescent CLSM (scale bar 50 μm).

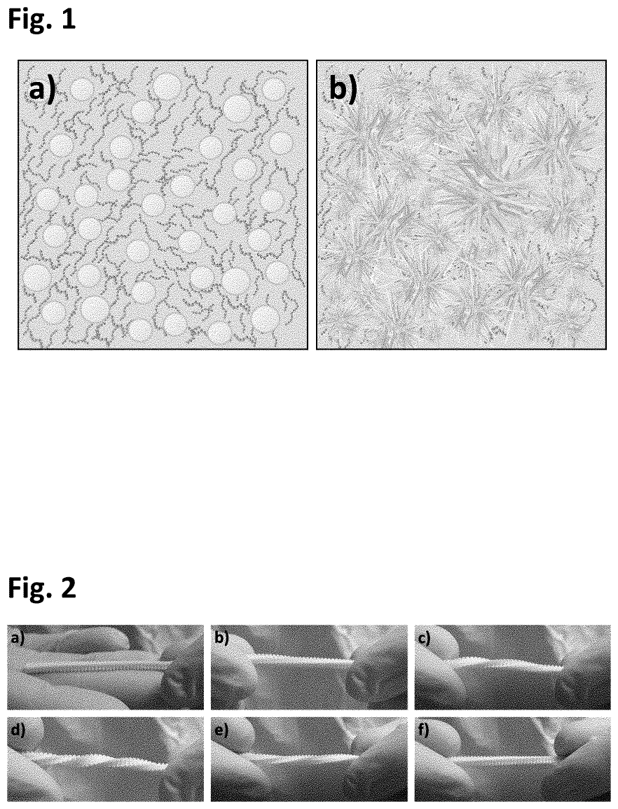

### DETAILED DESCRIPTION

All the terms as used herein in this application, unless otherwise stated, shall be understood in their ordinary meaning as known in the art. Other more specific definitions for certain terms as used in the present application are as set forth below and are intended to apply uniformly throughout the specification and claims unless an otherwise expressly set out definition provides a broader definition.

For the purposes of the present invention, any ranges given include both the lower and the upper end-points of the range.

As used herein, the meaning of the term “comprising” encompasses three alternatives, namely “comprising”, “consisting of” and “consisting essentially of”.

The present invention provides in a first aspect of the invention a method for preparing a 3D-printed bone graft.

In a first step, the method comprises the preparation of an ink composition comprising a binder solution and α-TCP particles.

### I. The Method of the Invention

### Step (a): Preparation of the Ink Composition

In one embodiment of the first aspect of the invention, the ceramic particles are added in the form of powder or dispersion.

In the context of the invention, the term “binder” encompasses polymers, oligomers and monomers.

In one embodiment of the first aspect of the invention, the ceramic particles are added to a solution comprising one or more non-water-soluble binders.

In the context of the present invention, the term “non-water-soluble”, means that the binder has a solubility in water lower than 10 mg/ml of water at 25° C.

Illustrative non-limitative examples of non-water-soluble binders include poly(hydroxy acids), polyesters (such as poly lactic acid (PLA), poly(L-lactic acid) (PLLA), poly glycolic acid (PGA), Poly lactic co-glycolic acid (PLGA), poly(L-lactic acid-co-glycolic acid) (PLLGA)), poly ε-caprolactone (PCL), and copolymers with polyethylene glycol (PEG); polyanhydrides, poly(ortho)esters, polyurethanes, poly(butyric acid), poly(valeric acid), poly(lactide-co-caprolactone), trimethylene carbonate, and the polymers described by Hubbell et al. in U.S. Pat. Nos. 5,654,381; 5,627,233; 5,628,863; 5,567,440; and 5,567,435, and which can be used in a variety of combinations, copolymers and blends thereof. In general, these materials degrade in vivo by both non-enzymatic and enzymatic hydrolysis, and by surface or bulk erosion.

In one embodiment the non-water-soluble polymeric binder(s) comprise(s) one or more polyester(s). In another embodiment, the non-water-soluble polymeric binder(s) comprise(s) one or more polyester(s) selected from the group consisting of polylactic acid (PLA), polyglycolic acid (PGA), copolymers of lactic acid and glycolic acid (i.e., polylactic-co-glycolic acid (PLGA)), and polycaprolactone (PCL).

In one embodiment the non-water-soluble non-water-soluble polymeric binder consists of a polyester(s) selected from the group consisting of polylactic acid (PLA), polyglycolic acid (PGA), copolymers of lactic acid and glycolic acid (i.e., polylactic-co-glycolic acid (PLGA)), and polycaprolactone (PCL). In another embodiment, the binder solution comprises PLLA, PLGA, PCL or a combination thereof.

In an alternative embodiment, the ceramic particles are added to an aqueous solution comprising one or more water-soluble photo-crosslinkable binder(s).

In the context of the present invention a photo-crosslinkable binder is considered to be “water soluble” when it is dissolved in an amount equal or higher than 10 mg/mL of water.

Illustrative non-limitative examples of water-soluble photo-crosslinkable binders suitable in the context of the invention are acrylates such as poly(ethylene glycol) diacrylate (PEGDA), Poly(ethylene glycol) dimethacrylate (PEGDMA)) and can contain functional groups such as methacrylates, dimethacrylates, triacrylates, and diacrylates, which can be used in a variety of combinations, copolymers and blends thereof and combination(s) of their pre-cursor monomers. Other synthetic oligomers that can be used are: polyanhydrides, polyorthoesters, poly(ester amides), polyamides, poly(ester ethers)polycarbonates, polyalkylenes such as polyethylene and polypropylene, polyalkylene terephthalates such as poly(ethylene terephthalate), polyvinyl ethers, polyvinyl esters such as poly(vinyl acetate), polysiloxanes, polystyrene (PS), polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutylacrylate), poly(octadecyl acrylate), and copolymers and mixtures thereof, polydioxanone and its copolymers, polyhydroxyalkanoates, poly(propylene fumarate), polyoxymethylene, and copolymers and blends thereof, as well as a variety of their combinations and combination(s) of their precursor monomers.

In one embodiment, the water-soluble photo-crosslinkable binder is PEGDA or PEGDMA.

When the binder solution comprises water-soluble photo-crosslinkable binder(s), it is required that the solution further includes a photoinitiator. The photoinitiator can be added during the preparation of the binder solution, simultaneously with the α-TCP particles or after adding the ceramic particles.

In the context of the invention, any photoinitiator already known in the state of the art is suitable. Illustrative non-limitative examples of these photoinitiators are: those comprise benzoins, including benzoin, benzoin ethers, such as benzoin methyl ether, benzoin ethyl ether and benzoin isopropyl ether, benzoin phenyl ether and benzoin acetate; those including acetophenones, including acetophenone, 2,2-dimethoxyacetophenone and 1,1-dichloroacetophenone; benzyl; benzyl ketals, such as benzyl dimethyl ketal and benzyl diethyl ketal; anthraquinones, including 2-methylanthraquinone, 2-ethylanthraquinone, 2-tert-butylanthraquinone, 1-chloroanthraquinone and 2-amylanthraquinone, triphenylphosphine; benzoylphosphine oxides, for example 2,4,6-trimethylbenzoyldiphenylphosphine oxide (Lucirin TPO), Phenyl-bis-(2,4,6-trimethylbenzoyl)-phosphinoxide (BAPO), Lithium phenyl-2,4,6-trimethylbenzoylphosphinate (LAP); benzophenones, such as benzophenone and 4,4′-bis(N,N′-dimethylamino)benzophenone; thioxanthones and xanthones; acridine derivatives; phenazine derivatives; quinoxaline derivatives or 1-phenyl-1,2-propanedione; 2-O-benzoyl oxime; 1-aminophenyl ketones or 1-hydroxyphenyl ketones, such as 1-hydroxycyclohexyl phenyl ketone, phenyl 1-hydroxyisopropyl ketone and 4-isopropylphenyl 1-hydroxyisopropyl ketone.

In one embodiment, the binder solution comprises PEGDA, a benzoylphosphine oxide (BAPO) and water.

In one embodiment, the binder solution comprises PEGDMA, a benzoylphosphine oxide (BAPO) and water.

The selection and amount of the appropriate photoinitiator forms part of the routine exercise of those skilled in the art.

The non-water-soluble binders are dissolved in appropriate organic solvents. In one embodiment, the non-water-soluble binder(s) are dissolved in a solvent which is liquid at 25° C. and at 760 mmHg, and has a vapour pressure at 25° C. equal or greater than 15 mmHg. Illustrative non-limitative examples are: methanol, ethanol, propanol, isopropanol, butanol, hexafluoroisopropanol (HFIP), carboxyl acids, sulfonic acids, formic acid, 1,4-Dioxane, tetrahydrofuran (THF), acetone, acetonitrile, dimethylformamide, dimethyl sulfoxide, hexane, benzene, toluene, diethyl ether, chloroform, ethyl acetate, dichloromethane, methylene chloride, oxolane and pyridine. In one embodiment, the non-water-soluble binder(s) are dissolved in 1,4-dioxane, dichloromethane, pyridine, chloroform, methylene chloride and, hexafluoroisopropanol (HFIP).

In another embodiment, the binder solution comprises the binder at % by weight, with respect to the total weight of the binder solution, from 5 to 80% w/w, particularly from 10 to 70% w/w.

In one embodiment the α-TCP is added to a binder solution, wherein the binder is a non-water-soluble binder, particularly a non-water-soluble polymeric binder comprising one or more polyesters, and it is at a % by weight with respect to the total weight of the binder solution from 5 to 60%. In one embodiment the α-TCP is added to a binder solution, wherein the binder is a non-water-soluble polymeric binder, particularly a non-water-soluble polymeric binder comprising one or more polyesters, and it is at a % by weight with respect to the total weight of the binder solution from 5 to 60%. In one embodiment the α-TCP is added to a binder solution, wherein the binder is a polyester and it is at a % by weight with respect to the total weight of the binder solution from 5 to 60%. In one embodiment the α-TCP is added to a binder solution, wherein the binder is a PLLA, PLGA, PCL or a combination thereof, and it is at a % by weight with respect to the total weight of the binder solution from 5 to 60%. In one embodiment the α-TCP is added to a binder solution, wherein the binder is PLLA, and it is at a % by weight with respect to the total weight of the binder solution from 5 to 40%. In one embodiment, the α-TCP is added to a binder solution, wherein the binder is PLGA, and it is at a % by weight with respect to the total weight of the binder solution from 15 to 60%. In one embodiment the α-TCP is added to a binder solution comprising PLLA at a % by weight with respect to the total weight of the binder solution from 5 to 40% dissolved in dicholoromethane (DCM) or 1,4-dioxane. In one embodiment the α-TCP is added to a binder solution comprising PLGA at a % by weight with respect to the total weight of the binder solution from 15 to 60%, dissolved in 1,4-dioxane.

In an alternative embodiment the water-soluble photo-crosslinkable binder is dissolved in water alone or in combination with another one or more water-soluble solvent(s).

In an alternative embodiment, the binder aqueous solution comprises, in addition to the photo-crosslinkable binder(s), one or more water-soluble binders other than those photo-crosslinkable. It is added to adjust the rheological properties (increasing printability during the 3D printing of the scaffolds), in order to have an adequate texture of the gel (binder solution). Illustrative non-limitative examples of these other water-soluble binders suitable to be added together with the photo-crosslinkable ones are the poly(oxypropylene)-poly(oxyethylene) copolymers, such as poloxamers, polyethylene glycols (PEG).

In one embodiment, the one or more water-soluble photo-crosslinkable binder(s) are in a higher % w/w with respect to the % w/w of the other water-soluble binder(s), the % w/w being with respect the total composition of the solution. In one embodiment, the one or more water-soluble photo-crosslinkable binder(s) are at a % w/w from 30 to 100% and the other water-soluble binder(s) are at a % w/w from 10 to 40 wt %.

In one embodiment, the weight ratio between the binder solution and α-TCP is from 0.1 to 2, particularly from 0.2 to 1.5, from 0.3 to 1.4, particularly the weight ratio is 0.2, 0.3, 0.4, 0.45, 0.5, 0.55, 0.6, 0.7, 0.8, 0.9, 1.0, 1.1, 1.2, 1.3, 1.4 or 1.5.

The “weight ratio”, in the context of the invention, is understood as the number of grams of the binder solution with respect to the number of grams of ceramic particles (α-TCP).

The binder solution in the self-setting ink may contain 10 to 60 g of the corresponding binder(s) (such as PLGA, PLLA, PCL) per 100 g total solution weight (i.e., intervals tested and that has been proved printable), preferably 15 to 55 g such as PLGA, PLLA, PCL) per 100 g total solution weight (i.e., optimized intervals for better printability, geometrical stability and mechanical properties).

In another embodiment of the invention, the α-TCP is added in the form of powder.

The α-TCP powder is sieved to particle sizes below 100 micrometers, particularly to particle sizes below 40 micrometers. The liquid to powder ratio is preferably between 0.2 and 0.7.

An amount of 0.2 to 1.7 g of binder solution may be used per g of α-TCP. Particularly an amount of 0.3 to 0.7 g of binder solution may be used per g of α-TCP. Particularly an amount of 0.4 to 0.6 g of binder solution may be used per g of α-TCP.

The resulting self-setting ink is stable at low temperatures (−80° C.), allowing the material to be stored. It also has a relatively low injection force at room temperature, ideally between 20 and 300 N, which allows the material to be injected during the printing process. It is also cohesive at room temperature in air.

In one embodiment of the invention, step (a) further comprises adding a minor amount of an hydroxyapatite compound. Illustrative non-limitative examples of suitable hydroxyapatite compounds are hydroxyapatite, dicalcium phosphate dihydrate, anhydrous dicalcium phosphate, tetracalcium phosphate, β-tricalcium phosphate, calcium-deficient hydroxyapatite, monocalcium phosphate monohydrate, mono-calcium phosphate, calcium pyrophosphate, precipitated hydroxyapatite, carbonated apatite (dahlite), octocalcium phosphate, amorphous calcium phosphate, oxyapatite, carbonatoapatite, magnesium oxide, phosphate salt, tricalcium silicate and calcium sulphate hydrate.

The amount of the above hydroxyapatite compound is that which allows to accelerate the later step of hydrolysis of the ceramic. It forms part of the routine activity of those skilled in the art to determine the more appropriate amount. Illustrative non-limitative suitable amounts of the hydroxyapatite compound can be in the range from 0.5% to 10% by weight with respect to the total weight of the ink composition.

In one embodiment of the invention, the ink composition is one as provided in Table 1:

TABLE 1

| Polymer(s) | Poly(ethylene glycol) diacrylate (PEGDA) | |
|---|---|---|
| | 400 g/mol and 600 g/mol, | |
| | Poly(ethylene glycol) dimethacrylate | |
| | (PEGDMA) 1K g/mol and 4K g/mol, | |
| | Poloxamer (Kolliphor 407) | |
| Photoinitiator | Phenylbis(2,4,6-trimethylbenzoyl) | |
| | phosphine oxide) (BAPO) | |
| Solvent | Water | |
| Photopolymer concentration | 30-100 | wt. % |
| in the binder solution | | |
| Poloxamer concentration | 0.10-40 | wt. % |
| in the binder solution | | |
| Photoinitiator volume | 0-10 | μl |

| | |
|---|---|
| Ceramic powder | α-tricalcium phosphate (α-TCP) |
| Liquid to powder ratio | 0.20-0.55 |
| (L/P) in the ink | |

provided that the sum of components is 100% w/w %.

In another embodiment, the ink composition is one as provided in Table 2:

TABLE 2

| Polymer(s) | Poly(L-lactic acid) (PLLA), | |
|---|---|---|
| | Poloxamer (Kolliphor 407) | |
| Solvent | Dichloromethane (DCM) | |
| PLLA concentration | 6-15 | wt. % |
| in the binder solution | | |
| Poloxamer concentration | 0%, 3-15 | wt. % |
| in the binder solution | | |

| | |
|---|---|
| Ceramic powder | α-tricalcium phosphate (α-TCP) |
| Liquid to powder ratio | 0.8-1.3 |
| (L/P) in the ink | |

provided that the sum of components is 100% w/w %.

When preparing the formulations of Table 2 above, it can be firstly prepared the water soluble binder (polaxamer with water). Separately, the non-water-soluble binder solution can also be prepared by dissolving the PLLA in DCM. Then, the small amount of the water-soluble binder is mixed with the non-water-soluble binder solution at the specific % wt.

In another embodiment, the ink composition is one as provided in Table 3:

TABLE 3

| | |
|---|---|
| Polymer(s) | Poly(L-lactic acid) (PLLA), |
| | Poly(lactic co-glycolic acid) (PLGA) |
| Solvent | 1,4-Dioxane |
| PLLA or PLGA concentration | 15-50 wt. % |
| in the binder solution | |
| Ceramic powder | α-tricalcium phosphate (α-TCP) |
| Liquid to powder ratio | 0.4-1.0 |
| (L/P) in the ink | |

provided that the sum of components is 100% w/w %.

The self-setting ink may be produced by a process comprising the following steps:
1. Dissolve the binder(s) in the solvent, until homogeneously dispersed and a viscous gel is obtained, which is referred to as the binder solution.
2. Add the photoinitiator to the binder solution (only if a photocurable binder is used) and mix until it is homogeneously dispersed within the binder solution.
3. Add the ceramic powder to the binder solution and mix until the ceramic particles are homogeneously dispersed, which is referred to as the ink. The mixing of the solid and liquid phase may require the use of high speed mixing techniques.

### Step b: 3D-Printing of the Bone Graft

3D printing step b) generally involves the configuration of the printer software (including the design of the graft from medical images) followed by the preparation of the printer, the preparation of the external material (including the ink, and the means by which it is placed in the printer injection system), and the printing process itself.

The printing process is generally performed by deposition of the ink in a defined pattern. The pattern fills the contour of the shape of a slice of the graft, generating a layer, and the superposition of those layers creates the three-dimensional shape. The shape may be pre-determined by a digitalised medical imaging technique and/or computer aided design.

Advantageously, these 3D constructs can be designed to mimic certain tissues and/or organs, including the osteochondral region of the articulate joint, and to have enhanced mechanical characteristics. In some embodiments, these fabricated 3D printed constructs can be subjected to surface modification, both with a chemically functionalized acetylated collagen coating and through absorption via poly-L-lysine coated carbon nanotubes so as to promote the growth and differentiation of MSCs.

One of the critical 3D scaffold design criteria for hard tissues is that they must have suitable mechanical properties. In addition, interconnected pores, specifically pore structures at the macro-scale, interconnected by smaller pores on a micro- and nano-scale are also indicative of the ECM of hard tissues, and are very important for hard tissue scaffold design. This sort of complicated, hierarchical structure is one that is difficult to recapitulate, if at all, and then more difficult to control in even very advanced electrospinning setups and other common scaffold fabrication techniques. With the advent of 3D printing, there is a possibility not only for the creation of delicate and intricate structures from the advanced working of strong and robust materials, but a potential to create highly ordered structures that could conceivably match any desired architecture [2]. This later advantage is one that also makes 3D printing attractive for other types of targeted tissue 3D scaffolds.

Currently, 3D printing uses a layered manufacturing method of printing thin depositions of material in a given pattern on top of previously printed material. This could allow for large, macro-scale objects that have complex, user-defined internal features, mimicking the architecture of a given organ. This could also allow for materials to be printed which encapsulate living cells into the artificial organ construct, creating a complex network of cells with an advantageous architecture conducive to organ function and cell/tissue growth.

Moreover, one of the most important challenges facing 3D construct design is vascularization. Scaffolds seeded with cells that begin to mature and form tissue have problems with the transportation of nutrients and essential signalling chemicals and growth factors, as well as removal of waste products within the internal structure of the scaffold. In the body, vascular networks accomplish these tasks, but new and under- formed vasculature present a daunting limitation to scaffold-based tissue repairs.

However, if a scaffold can be fabricated with designed transport channels and structures that mimic vascularized tissue, then it could be possible to ameliorate this limitation. 3D printing presents a potential ability to accomplish this because, as stated previously, it is possible to create structures with predesigned complex, macro-scale internal architectures.

One of ordinary skill in the art can recognize that the use of the techniques and methods described herein can be applied towards the generation of 3D printed constructs that mimic a variety of tissues and/or biological environments. In addition, one of ordinary skill in the art can appreciate that these constructs can also be modified to include surface modifications (or other modifications not exclusive to the surface) that can more appropriately mimic the native tissue or environment with which they are intended to interact. In addition, one of ordinary skill in the art can readily appreciate that these constructs can be further modified to more specifically and/or efficiently promote the differentiation, growth, and/or production of cells and tissues specific to a particular biological environment and/or organ.

### Step (c): Providing Cohesion to the 3D-Printed Scaffold

In this step the hardening of the binder component occurs, reducing the solvent content, and providing a continuous phase (matrix) within the dispersed α-TCP particles (cohesion between the matrix and the particles).

There are well-known techniques suitable to promote the cohesion of the 3D-printed scaffold. Thus, the binder phase can either be hardened/consolidated through evaporation/sublimation or dissolution of the solvent (e.g., at room temperature, drying the parts in a stove, dissector or freeze drying to guarantee the complete release/evaporation of the solvent), or be cured by photo-polymerization (e.g. by exposure to UV radiation), depending on the particular binder(s).

In one embodiment, the binder solution comprises non-water-soluble binders, as defined in any of the above embodiments, and step (c) comprises evaporating the solvent. The evaporation can be performed, for instance, at room temperature, or, alternatively, drying the parts in a stove, dissector or freeze drying. In the examples provided below, the evaporation of the scaffolds is performed by evaporation of the solvent in ambient conditions at room temperature and pressure. This step was performed in a clean room ISO-7. The particular conditions can be routinary determine by those skilled in the art.

Alternatively, the hardening is performed by crosslinking the water-soluble photocurable binder(s) in the presence of the one or more photoinitiator(s). In this embodiment the resulting scaffold will comprise a photo-crosslinked polymeric matrix.

The terms “cured polymer” and “crosslinked polymer” have the same meaning, can be used interchangeably and refers to a polymer wherein different polymeric chains (such as oligomers), which can be linear or branched, or monomers are linked through at least covalent bonds.

The term “monomer” means, as recognized by IUPAC, a molecule that has one or more polymerizable end-groups that can undergo polymerization thereby contributing constitutional units to the essential structure of a macromolecule.

In the present invention, the terms “cured”/“curing” and “crosslinked”/“crosslinking” have the same meaning and can be used interchangeably. The term “curing” refers to the toughening or hardening of a polymer material by cross-linking of polymer chains, brought about by cross-linker agents such as commercially available chemical additives, ultraviolet radiation, electron beam or heat. In this process the polymer viscosity drops initially upon the application of heat, passes through a region of maximum flow and begins to increase as the chemical reactions increase the average length and the degree of cross-linking between the constituent polymers. This process continues until a continuous 3-dimensional network of polymer chains is created—this stage is termed gelation.

In one embodiment, the hardening is performed by crosslinking water-soluble photocurable binder(s) in the presence of a photoinitiator and using exposure under UV-light.

The suitable water-soluble photocurable binder(s), as well as the photoinitiator(s), are as defined above.

### Step (d): Hardening of the Ceramic Phase

The hardening of the ceramic phase gives rise to the hydrolysis of the α-TCP contained in the self-setting ink, which transforms into calcium deficient hydroxyapatite (CDHA) according to the following reaction:

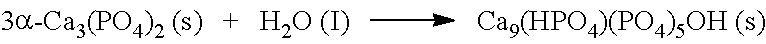

these CDHA crystals interlocking to provide a continuous ceramic phase. When performed in an autoclave, β-TCP, a polymorph of α-TCP may also be generated. The amount of β-TCP increases with pressure.

Performing step (d) a crystalline phase very similar to the mineral phase of bone, especially if carbonate is incorporated into CDHA during the hardening process, is obtained. It can be accelerated with temperature and pressure, which slightly changes the structure, but preserves the high specific surface area; guaranteeing the micro and nano porosity that is necessary for adequate biological response in vivo.

In one embodiment hardened by immersion in water or an aqueous solution. The aqueous solution may contain ions that can be incorporated into the calcium-deficient hydroxyapatite (CDHA). Thus the ions may be incorporated into the scaffold while the scaffold is hardening.

The hardening step may be performed within few hours or days. In one embodiment step (d) is performed for a period of time of 120 minutes or less, particularly from 40 to 80 minutes, particularly from 45 to 65 minutes, particularly for 55 min.

In one embodiment the aqueous solution consists of water.

In another embodiment, the aqueous solution consists of water and one or more ions. The ions in the aqueous solution may be selected from anions such as carbonate, bicarbonate, silicate, and/or cations such as Ca, Mg, Sr, Ce, Al, Zn, Ag, Co, Cu and other transition metals. These ions can be incorporated in the CDHA during its precipitation while the process of hydrolysis of α-TCP occurs. In this way, the ion doping process is simultaneous to the CDHA formation, and it allows the shape and geometry of the printed scaffold to be maintained. For example, CDHA could be doped with carbon ions, by immersing the scaffolds in an aqueous solution containing 25 g sodium bicarbonate dissolved in 1 L of water. The hardening of the ceramic phase then may take place at room temperature and atmospheric pressure, physiological conditions or in an autoclave (as in the examples).

The hardening step d) may be performed by immersing the scaffold in water or an aqueous solution at a temperature of 0 to 100° C. In one embodiment step (d) is performed at a temperature above 50° C., above 60° C., above 70° C., above 80° C. or above 90° C. In another embodiment step (d) is performed at 100° C.

In one embodiment step (d) is performed by immersing the scaffold in water and heating at a temperature above 50° C., above 60° C., above 70° C., above 80° C. or above 90° C. In another embodiment step (d) it is immersing in water and heating at 90-100° C., particularly at 100° C.

Alternatively, the hardening step may be performed by immersing the scaffold in water and autoclaving at a temperature at or above 100° C., for example at a temperature in the range of 100 to 170° C., at a temperature in the range of 100 to 150° C., at a temperature in the range of 100 to 130° C., at a temperature in the range of 110 to 130° C., at a temperature in the range of 115 to 125° C.

The hardening step may be performed by immersing the scaffold in an aqueous solution consisting of water and one or more ions and autoclaving at a temperature at or above 100° C., for example at a temperature in the range of 100 to 170° C., at a temperature in the range of 100 to 150° C., at a temperature in the range of 100 to 130° C., at a temperature in the range of 110 to 130° C., at a temperature in the range of 115 to 125° C.

The hardening step (d) may be performed by immersing the scaffold in water and autoclaving at a pressure at or above 1 atm of absolute pressure, for example at a pressure in the range of 1 atm to 4 atm, at a pressure in the range of 1 atm to 3 atm, at a pressure in the range of 0.5 atm to 2 atm, particularly from 0.5 to 1.5 atm, particularly at 1 atm., of absolute pressure.

Alternatively, the hardening step (d) may be performed by immersing the scaffold in an aqueous solution including ions, and autoclaving at a pressure at or above 1 atm of absolute pressure, for example at a pressure in the range from 1 atm to 4 atm, at a pressure in the range from 1 atm to 3 atm, at a pressure in the range from 0.5 atm to 2 atm, particularly from 0.5 to 1.5 atm, particularly at 1 atm., of absolute pressure.

In one embodiment, step (d) is performed at a temperature equal or above 90° C., particularly equal or above 100° C., at a pressure from 0.5 to 4 atm., of absolute pressure.

In another embodiment, step (d) comprises immersing the scaffold in water and autoclaving at a temperature equal or above 90° C., particularly equal or above 100° C., at a pressure from 0.5 to 4 atm., of absolute pressure. In another embodiment, step (d) comprises immersing the scaffold in water and autoclaving at a temperature around 100° C., and a at pressure from 0.5 to 2 atm, particularly from 0.5 to 1.5 atm, particularly at 1 atm., of absolute pressure.

In another embodiment, step (d) comprises immersing the scaffold in water and heating at a temperature equal or above 90° C., particularly equal or above 100° C., and a at pressure from 0.5 to 4 atm., of absolute pressure for 120 minutes or less.

In another embodiment, step (d) comprises immersing the scaffold in water and heating at a temperature above 50° C., and a at pressure from 0.5 to 2 atm, particularly from 0.5 to 1.5 atm, particularly at 1 atm., of absolute pressure for 120 minutes or less. In another embodiment, step (d) comprises immersing the scaffold in water and heating at a temperature equal or above 90° C., particularly equal or above 100° C., and a at pressure from 0.5 to 4 atm., of absolute pressure for 60 minutes or less. In another embodiment, step (d) comprises immersing the scaffold in water and heating at a temperature around 100° C., and a at pressure from 0.5 to 2 atm, particularly from 0.5 to 1.5 atm, particularly at 1 atm., of absolute pressure for 60 minutes or less.

In another embodiment, step (d) is comprises immersing the scaffold in water at a temperature from 90 to 110° C., for 45 to 65 minutes, at 0.5 to 1.5 atm.

In another embodiment, step (d) comprises immersing the scaffold in water at a temperature of 100° C., for 55 minutes, at 1 atm, absolute pressure.

In another embodiment, step (d) comprises immersing the scaffold in an aqueous solution comprising ions, and heating at a temperature above 50° C., and a at pressure from 0.5 to 2 atm, particularly from 0.5 to 1.5 atm, particularly at 1 atm., of absolute pressure for 120 minutes or less. In another embodiment, step (d) comprises immersing the scaffold in an aqueous solution comprising ions, and heating at a temperature equal or above 90° C., particularly equal or above 100° C., and a at pressure from 0.5 to 4 atm., of absolute pressure for 60 minutes or less. In another embodiment, step (d) comprises immersing the scaffold in an aqueous solution comprising ions, and heating at a temperature around 100° C., and a at pressure from 0.5 to 2 atm, particularly from 0.5 to 1.5 atm, particularly at 1 atm., of absolute pressure for 60 minutes or less.

In another embodiment, step (d) is comprises immersing the scaffold in an aqueous solution comprising ions, at a temperature from 90 to 110° C., for 45 to 65 minutes, at 0.5 to 1.5 atm.

In another embodiment, step (d) comprises immersing the scaffold in an aqueous solution comprising ions, at a temperature of 100, for 55 minutes, at 1 atm, absolute pressure.

The reaction conditions will determine the final composition of the crystalline phase created during step (d). Ideally, the crystalline phase should be 100% CDHA crystals. But embodiments of the invention are also those wherein the crystalline phase includes α-TCP and/or β-TCP.

In an alternative embodiment, the hardening of the ceramic phase may occur in situ once implanted in the patient's body, when in contact/wettened with the body fluid (i.e. at physiological conditions). Avoiding the ceramic consolidation step before implantation of the scaffold leads to a curious property, namely presenting a flexible scaffold pre-implantation, which consolidates and hardens over time once in contact with the body fluid after implantation, resulting in a transformation in situ from a flexible to a rigid scaffold. In this case, the washing step may be performed in a way that avoids starting the consolidation reaction/process of the ceramic phase in the scaffold.

### Optional Additional Steps:

The method may comprise one or more additional step(s) after hardening step d).

In one embodiment, the additional step comprises coating the scaffold resulting from step (d) with a binder solution as defined in step (a) according to the first aspect and any of the embodiments provided herein above.

The skilled person can routinary optimize the concentration of the binder's solution and use any of the well-known techniques known in the state of the art. An illustrative way is provided below, wherein the coating is performed by immersing the scaffold in the binder solution previously prepared.

In one embodiment, the coating is made with a solution including the same binder as the one used in the binder solution referred in step (a) of the method of the invention. In an alternative embodiment, the coating is made with a solution including other binder than the one used in the binder solution referred in step (a) of the method of the invention.

In another embodiment, the additional step is a washing step which may be performed by immersing the hardened scaffold in water. The additional step has the effect of further removing the solvent (in the case of non-water-soluble binders) or un-cross-linked matters (e.g., monomers, oligomers, polymers, photoinitiator) from the scaffold.

In one embodiment, the washing step comprises immersing the scaffold in water, preferably distilled water, at room temperature (from 15 to 25° C., for instance). The sample may be immersed in water for short periods of time of about 1 to 30 minutes, for instance.

### Optional Combined Hardening and Washing Step:

In one embodiment, hardening step d) and the optional washing step may be carried out simultaneously. This may be achieved by periodically changing the immersing solution during hardening step d). For example, the hardening step may be carried out for a certain period of time, stopped, the immersing solution changed and the hardening step started again. This process can be repeated a number of times and will have the effect of washing the scaffold during the hardening step.

In one embodiment, the immersing solution can be changed 2 to 5 times during hardening step d), preferably 2 to 3 times. The immersing solution can be changed at regular intervals or irregular intervals.

The immersing solution may be water or an aqueous solution. The aqueous solution employed may contain one or more ions. These ions may be selected from anions such as carbonate, bicarbonate, silicate, and/or cations such as Ca, Mg, Sr, Ce, Al, Zn, Ag, Co, Cu, and other transition metals.

Preferably the one or more ions are selected from the group consisting of carbonate, bicarbonate, silicate, calcium, strontium, zinc, cobalt and copper.

In an alternative embodiment, step (d) wherein step (d) comprises subjecting the bone graft resulting from step (c) to water vapor atmosphere. This step can be performed, for instance, in autoclave (without immersing the scaffold in water solution), heating to 100 degrees, at 1 atm of absolute pressure. Or just in contact with humid atmosphere (such as the humidity found in the air at room temperature).

In one embodiment the method of the first aspect of the invention comprises:
(a) preparing an ink composition comprising 10-60 wt % of PLGA dissolved in 1,4-dioxane, and α-TCP at a weight ratio binder solution:α-TCP from 0.4 to 0.7;
(b) 3D-printing;
(c) evaporating the 1,4-dioxane; and
(d) immersing the scaffold in water at 100° C., 1 atm, for less than 60 min, particularly at 55 min.

### II. The Scaffold of the Invention

The inventors of the present invention surprisingly have found that it is possible to produce 3D-printed personalized synthetic bone grafts using a composite self-setting ink containing reactive ceramic particles in an organogel binder or photopolymerizable solution, that after hardening results in a ceramic matrix intertwined with polymeric fibers. The ceramic phase, which is obtained by a dissolution-precipitation process, is more similar to bone mineral than prior art 3D-printed synthetic grafts, which are consolidated by sintering at high temperatures.

The resulting composite scaffold has a mineral phase closer to bone, and a polymeric phase rendering the scaffold more though (mimicking the mechanical function of the collagen fibrils in native bone), which makes the scaffold more biocompatible than sintered scaffolds and having enhanced fracture toughness compared to a pure ceramic scaffold hardened at low temperatures. Moreover, as the scaffold is composed of a ceramic mineral phase close to bone, entangled with a polymeric biocompatible phase, the scaffold is more similar to the native bone compared to other synthetic scaffolds. The scaffold may be used as a synthetic bone graft.

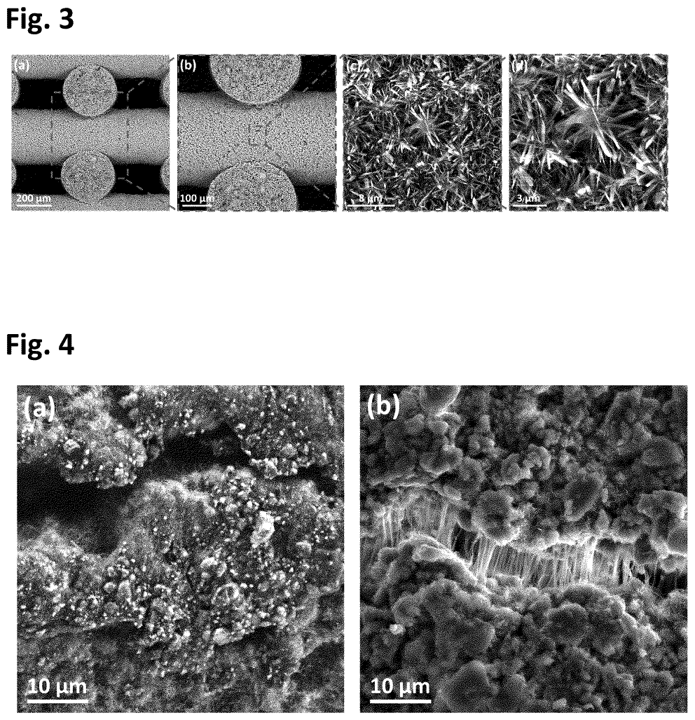

In the context of the present invention, the term “3D-printed” when referring to the scaffold of the invention is one resulting from any of the available 3D printing techniques. The use of 3D printing confers to the resulting product one or more of the following exclusive and inherent technical features: repetition of a defined pattern; homogeneous distribution of the macropores (as a consequence of the repeating pattern); interconnectivity of the macropores, forming tunnels (such as shown, for instance in FIG. 3).

In the context of the invention “continuous phase” and “matrix” have the same meaning and are used interchangeably.

In the context of the invention, when reference is made to a “matrix made from” a water-soluble or non-water-soluble binder, it has to be understood as a matrix which is prepared starting from the water- or non-water-soluble binder(s).

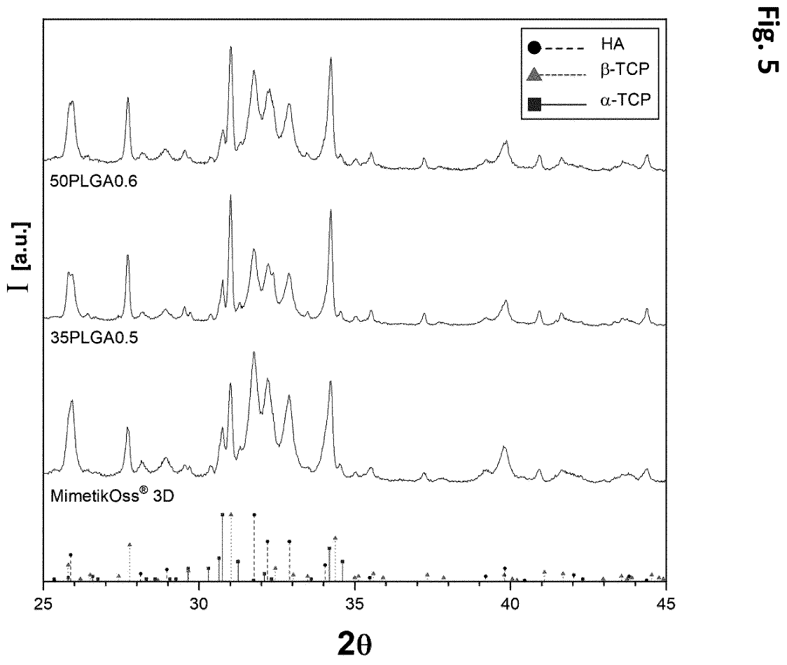

As it has been stated above, the bone graft of the invention is characterized by incorporating two matrixes (continuous phases), one ceramic including a crystalline phase and another, forming filaments, corresponding to the binder matrix. Both matrixes are in admixture, so when a SEM image is taken the skilled person can see that both matrixes are intertwined (see FIG. 4(b)). FIG. 4 (a) includes the SEM images of the comparative bone graft used in the examples, which is a pure ceramic scaffold: no binder fibers (i.e., no binder matrix) are observed.

As it has been stated above, the ceramic matrix comprises a crystalline phase including interlocked CDHA crystals. This crystalline phase, which can be easily identified by image techniques such as SEM (FIG. 3 (a)-(d)), can further include crystals of β-TCP and/or α-TCP.

In the context of the present invention, the term “crystalline phase” of the ceramic matrix corresponds to CDHA crystals and, optionally, to α-TCP and/or β-TCP crystals, if they are present. In order to determine the amount of CDHA, α-TCP and β-TCP, an X-ray powder diffraction can be performed of the powder after manually crushing the scaffold in an agate mortar. The diffractometer (D8 Advance, Bruker) equipped with a Cu Ka X-ray tube was operated at 40 kV and 40 mA. Data were collected in 0.02 steps over the 3 h range of 10-80 with a counting time of 3 s per step. Phase quantification was performed using the reference intensity ratio method (EVA, Bruker) comparing diffraction patterns of the crystalline structures of α-TCP (ICDD PDF 01-070-0364), CDHA (ICDD PDF 01-086-1201) and β-TCP (ICDD PDF 01-070-2065). According to the information provided in International Centre for Diffraction Data: ICDD (https://www.icdd.com/), each crystallin phase/material has a specific reference number in the database of ICDD PDF. The calculation of the crystalline component was performed by the EVA. It is a well known referencing system for identifying crystalline phases by x-ray diffraction. These patrons are used as references when identifying peaks in the DRX spectra of your sample.

In one embodiment, the crystalline phase of the ceramic matrix consists of CDHA crystals (i.e., the crystalline phase is 100% interlocked CDHA). In an alternative embodiment, the crystalline phase comprises, in addition to CDHA crystals, α-TCP and/or β-TCP crystals.

In one embodiment, the crystalline phase of the ceramic matrix comprises CDHA crystals at a percentage by weight from 30 to 100%, particularly from 40 to 75%, or particularly from 60 to 70% with respect to the total weight of the crystalline phase of the ceramic matrix.

In another embodiment, the crystalline phase of the ceramic matrix comprises, in addition to the CDHA crystals, α-TCP crystals at a weight percentage from 5 to 25%, particularly from 10 to 20%, with respect to the total weight of the crystalline phase of the ceramic matrix.

In another embodiment, the crystalline phase of the ceramic matrix comprises, in addition to the CDHA crystals, β-TCP crystals at a weight percentage from 15 to 30%, particularly from 18 to 25%, with respect to the total weight of the crystalline phase of the ceramic matrix.

In another embodiment, the ceramic matrix comprises a crystalline phase with the following composition:

$$β - TCP : 20 - 20.2 \% ;$$

$$α - TCP : 12.4 - 1 5.3 \%$$

$$CDHA : 64.4 - 67.5 \%$$

wherein the percentages by weight are determined with respect to the total weight of the crystalline phase of the ceramic matrix, and the sum of the components provides 100%.

In the context of the invention, there is no particular requirement in the morphology of CDHA crystals. They can acquire a needle-like morphology (with intertwined polymer fibers as determined by Field Emission—Scanning Electron Microscopy (FIG. 3), or a plate-like one, for instance. The only requirement is that the CDHA crystals are interlocked to create such matrix. The interlocking does not require the creation of ionic or covalent interactions but the physical contact between the crystals.

In one embodiment, the CDHA crystals are doped with one or more groups selected from the group consisting of carbonate, bicarbonate, silicate, Ca, Mg, Sr, Ce, Al, Zn, Ag, Co, Cu and other transition metals.

The ceramic matrix and the binder matrix are in admixture. In the context of the invention the expression “in admixture” means that the two matrixes are mixed together to provide a substantially homogeneous composition.

All the embodiments provided above regarding the water-soluble and non-water-soluble binders used in the method, are also embodiments of the resulting bone grafts.

In one embodiment, the binder matrix comprises one or more polyester binders.

In another embodiment, the binder matrix comprises one or more polyesters selected from PLA, PLLA, PGA, PLGA, PCL and any combination thereof.

In an alternative embodiment, the binder matrix comprises one or more photo-crosslinked binder(s), and includes a photoinitiator.

In one embodiment the photocrosslinked polymers are polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutylacrylate), poly(octadecyl acrylate), and copolymers and mixtures thereof, polydioxanone and its copolymers, polyhydroxyalkanoates, poly(propylene fumarate), polyoxymethylene, and copolymers and blends thereof, as well as a variety of their combinations and a combination of their precursor monomers.

In one embodiment, the polymeric matrix comprises photocrosslinked PEGDA or PEGDMA.

In one embodiment, the polymeric matrix comprises photocrosslinked PEGDMA.

In an alternative embodiment, the binder matrix can comprise, in addition to the photo-crosslinkable binder(s), one or more water-soluble binders other than those photo-crosslinkable. Illustrative non-limitative examples of these other water-soluble binders suitable to be added together with the photo-crosslinkable ones are the poly(oxypropylene)-poly(oxyethylene) copolymers, such as poloxamers, polyethylene glycol (PEG).

In one embodiment, the one or more water-soluble photo-crosslinkable binder(s) are in a higher % w/w with respect to the % w/w of the other water-soluble binder(s), the % w/w being with respect the total composition. In one embodiment, the one or more water-soluble photo-crosslinkable binder(s) are at a % w/w from 30 to 100% and the other water-soluble binder(s) are at a % w/w from to 40 wt %.

In one embodiment, the weight ratio between the binder(s) and α-TCP is from 0.1 to 2, particularly from 0.2 to 1.5, from 0.3 to 1.4, particularly the weight ratio is 0.2, 0.3, 0.4, 0.5, 0.6, 0.7, 0.8, 0.9, 1.0, 1.1, 1.2, 1.3, 1.4 or 1.5.

In the context of the invention, any photoinitiator already known in the state of the art is suitable. Illustrative non-limitative examples of these photoinitiators are: those comprise benzoins, including benzoin, benzoin ethers, such as benzoin methyl ether, benzoin ethyl ether and benzoin isopropyl ether, benzoin phenyl ether and benzoin acetate; those including acetophenones, including acetophenone, 2,2-dimethoxyacetophenone and 1,1-dichloroacetophenone; benzyl; benzyl ketals, such as benzyl dimethyl ketal and benzyl diethyl ketal; anthraquinones, including 2-methylanthraquinone, 2-ethylanthraquinone, 2-tert-butylanthraquinone, 1-chloroanthraquinone and 2-amylanthraquinone, triphenylphosphine; benzoylphosphine oxides, for example 2,4,6-trimethylbenzoyldiphenylphosphine oxide (Lucirin TPO), Phenyl-bis-(2,4,6-trimethylbenzoyl)-phosphinoxide (BAPO), Lithium phenyl-2,4,6-trimethylbenzoylphosphinate (LAP); benzophenones, such as benzophenone and 4,4′-bis(N,N′-dimethylamino)benzophenone; thioxanthones and xanthones; acridine derivatives; phenazine derivatives; quinoxaline derivatives or 1-phenyl-1,2-propanedione; 2-O-benzoyl oxime; 1-aminophenyl ketones or 1-hydroxyphenyl ketones, such as 1-hydroxycyclohexyl phenyl ketone, phenyl 1-hydroxyisopropyl ketone and 4-isopropylphenyl 1-hydroxyisopropyl ketone.

In one embodiment, the binder matrix comprises photo-crosslinked PEGDA and a benzoylphosphine oxide, such as BAPO or TPO.

In one embodiment, the binder matrix comprises photo-crosslinked PEGDMA and a benzoylphosphine oxide, such as BAPO or TPO.

In another embodiment, the binder matrix is at a weight % from 5 to 30% with respect to the total weight of the composition, particularly from 7 to 30% w/w.

The composition of the synthetic bone graft may have pores of less than 1 μm, as determined by mercury intrusion porosimetry.

In another embodiment, the synthetic bone graft has nano-micro porosity in the range of 0.1% to 30%, particularly from 0.1 to 15%.

In a further embodiment, the synthetic bone graft has macro-porosity in the range of 10% to 80%. In a further embodiment, the synthetic bone graft has macro-porosity in the range of 30% to 70%. In yet a further embodiment, the synthetic bone graft has a total porosity in the range of 40% to 60%.

Macro-porosity may be determined by calculating the difference between the total porosity and the nano-micro porosity, according to the following formula:

$${P}_{m a c r o} ( \% ) = {P}_{T O T} ( \% ) - {P}_{m i c r o} ( \% )$$

where the nano-micro porosity is determined by mercury intrusion porosimetry. The total porosity may be determined using the following equation:

$${P}_{T O T} ( \% ) = ( 1 - \frac{{ρ}_{app} [ \frac{g}{{cm}^{3}} ]}{{ρ}_{s kel} [ \frac{g}{{cm}^{3}} ]} ) \cdot 100$$

where skeletal density of the scaffolds (ρₛₖₑₗ) was assessed by helium pycnometry. The apparent density of the scaffolds (ρₐₚₚ) was calculated as the quotient of the scaffold mass over the scaffold equivalent cubic volume obtained from the measurements of the scaffold length, width and height.

Macroporosity can also be estimated by microcomputed tomography (micro-CT). Micro-CT uses x-rays to create cross-sections of a physical object that can be used to recreate a virtual model (3D model) without destroying the original object. The prefix micro- is used to indicate that the pixel sizes of the cross-sections are in the micrometre range.

The value measured by micro-CT corresponds to the average pore size whereas the value measured by mercury intrusion porosimetry provides the average entrance size of the macropores.

The composition of the synthetic bone graft may have a total porosity in the range of 20% to 80%, as determined by mercury intrusion porosimetry. Preferably the composition of the synthetic bone graft has a total porosity in the range of 60% to 80% as determined by mercury intrusion porosimetry; most preferably the total porosity is in the range of 68% to 80% as determined by mercury intrusion porosimetry. The total porosity may be determined using the following equation:

$${P}_{T O T} ( \% ) = ( 1 - \frac{{ρ}_{app} [ \frac{g}{{cm}^{3}} ]}{{ρ}_{s kel} [ \frac{g}{{cm}^{3}} ]} ) \cdot 100$$

where skeletal density of the scaffolds (ρₛₖₑₗ) was assessed by helium pycnometry. The apparent density of the scaffolds (ρₐₚₚ) was calculated as the quotient of the scaffold mass over the scaffold equivalent cubic volume obtained from the measurements of the scaffold length, width and height.

The composition of the synthetic bone graft may show a needle-like or a plate-like morphology as determined by Field Emission-Scanning Electron Microscopy. The composition of the synthetic bone graft preferably shows a needle-like morphology as determined by Field Emission-Scanning Electron Microscopy.

In another embodiment, the synthetic bone graft has nano-micro porosity in the range of 0.1% to 30%, particularly from 0.1 to 15%.

In a further embodiment, the synthetic bone graft has macro-porosity in the range of 10% to 80%. In a further embodiment, the synthetic bone graft has macro-porosity in the range of 30% to 70%. In yet a further embodiment, the synthetic bone graft has a total porosity in the range of 40% to 60%.

The composition of the synthetic bone graft may have an apparent density below 2 g/cm, particularly below 1.5 g/cm, as determined by the quotient of the scaffold mass over the scaffold equivalent cubic volume obtained from the measurements of the scaffold length, width and height.

The composition of the synthetic bone graft may have a skeletal density in the range of 2.30 to 3.14 g/cm³, as determined by helium pycnometry.

The composition of the synthetic bone graft may have specific surface area (SSA) in the range of 1 to 15 m²/g, as determined by nitrogen adsorption and BET analysis. Preferably the composition of the synthetic bone graft has a specific surface area (SSA) in the range of 1 to 10 m²/g, as determined by nitrogen adsorption and BET analysis.

In another embodiment, the present invention relates to a synthetic bone graft with a composition comprising calcium-deficient hydroxyapatite (CDHA) in an amount of at least 60 wt. % with respect to the total weight of the composition, optionally α-TCP and/or β-TCP, wherein the synthetic bone graft has a specific surface area (SSA) in the range of 2 to 8 m²/g.

The composition of the synthetic bone graft may have a flexural strength of 1 to 8 MPa and a flexural toughness of 4 to 80 Jm-2. Determined on 3D-printed bars of 50 mm in length, 4 mm of width and 3 mm of height (50×4×3 mm3, coinciding with the printer X, Y and Z axis, respectively), printed with orthogonal pattern, nozzle inner diameter 24 Ga-25 Ga (0.260-0.311 mm), layer height (0.2-0.23 mm) and strand separation (250 μm). Determined by monotonic uniaxial compressive loading in the z-direction (i.e., three-point bending) using a hybrid rheometer (TA Instruments, Discovery HR-2, Germany) equipped with a 3-point bending clamp with a span of 40 mm. The test was run until fracture under displacement-control mode at a crosshead speed of 0.5 mm·min⁻¹. Twelve 3D-printed samples per condition and the samples were tested in wet conditions (immersed in phosphate-buffered saline solution at 37° C. for 12h). Tested according to ASTM C 1161-02c. (Table 5 and Table 6).

The composition of the synthetic bone graft may have a compressive strength of 10 to 30 MPa and a compressive modulus of 100 to 300 MPa. Determined 3D-printed rectangles of 6 mm in cross section and 9 mm in height (6×6×9 mm3, coinciding with the printer X, Y and Z axis, respectively), printed with orthogonal pattern, nozzle inner diameter 25 Ga (0.260 mm), layer height (0.2 mm) and strand separation (250 μm). Determined by monotonic uniaxial compressive loading in the z-direction (Bionix servo-hydraulic test system, MTS Systems, MN, USA). The test was run until fracture under displacement-control mode at a crosshead speed of 0.5 mm·min⁻¹. Twelve 3D-printed samples per condition and the samples were tested in wet conditions (immersed in phosphate-buffered saline solution at 37° C. for 12h). Tested according to ISO 13175-3.

In the context of the invention, the term “obtainable” and “obtained” have the same meaning and are used interchangeably. In any case, the expression “obtainable” encompasses the term “obtained”.

### Applications

The composition comprising calcium-deficient hydroxyapatite (CDHA) described herein is biomimetic and structurally (on a macro and micro scale) similar to the mineral phase of bone. This makes it a particularly suitable material for bone grafting and thus for use as a synthetic bone graft.

The synthetic bone graft described herein may be used in the following locations in a body:
cranio-maxillo facial/dental: sinus lift, alveolar filling, peri-implantation filling, affixed graft,
inlay/onlay graft, maxillary distraction. cranio-facial plastic. orbital floor
cranium reconstruction
orthognathics
spine: spine fusion, spinal cage filling
Iliac crest harvesting
upper limb: humeral head fracture, glenoidal prosthesis, humeral fracture, distal radius
fracture
lower limb: femoral head fracture, femoral nail revision, screws ablation, hip prosthesis,
knee prosthesis, tumoral filling, tibial fracture
foot: calcaneum fracture, phalanx fusion, talus fusion,
cranium reconstruction and
orthognathics.

### EXAMPLES

The invention is illustrated with the following non-limiting examples.

In order to obtain an optimized ink, with the desired rheological properties for a good printability and geometrical stability, different compositions and concentrations have been tested, such as different polymer concentrations in the binder solution, different powder to liquid ratio (i.e., quantity of ceramic powder in the ink), different amount of photo initiator in the ink and combinations of different polymers in the binder solution, as well as different solvents. The intervals tested are stated bellow.

The scaffolds were 3D-printed using a custom-made extrusion-based 3D printing machine and the scaffolds were printed with orthogonal pattern, nozzle inner diameter 25 Ga (0.260 mm), layer height (0.2 mm), strand separation (250 μm) and with a deposition speed of 10 mm/s.

### Example 1. Printable Self-Setting Inks Based on Water-Soluble Photocurable Polymers

3D-printed scaffolds containing binder solution (50% PEGDA 400 g/mol, 25% Poloxamer (Kolliphor 407), 10 μl BAPO, water), and α-TCP in a L/P=0.55, resulting in 21.57 wt. % polymeric phase with respect to the total weight of the final composition. Curing of the polymeric phase by exposure of UV-light (380 nm) for 2×30 min. Hardening of the ceramic phase by immersion in water at 100° C., 1 atm, for 15 min.

### Example 2. Printable Self-Setting Inks Based on Polymers Soluble in Dichloromethane (DCM)

### Example 2.1

3D-printed scaffolds containing binder solution (12% PLLA, DCM), and α-TCP in a L/P=1, resulting in 10.71 wt. % polymeric phase with respect to the total weight of the final composition. Hardening of polymeric phase by evaporation of solvent (DCM) at room temperature and pressure. This step was performed in a clean room ISO-7 (temperature range between 17° C.-26° C. and a pressure range between 2.0-5.0 mm H2O (20 Pa)). Hardening of the ceramic phase by immersion in water at 100° C., 1 atm, for 55 min.

### Example 2.2

3D-printed scaffolds containing binder solution (12% PLLA, DCM), and α-TCP in a L/P=0.8, resulting in 8.76 wt. % polymeric phase with respect to the total weight of the final composition. Hardening of polymeric phase by evaporation of solvent (DCM) at room temperature and pressure. This step was performed in a clean room ISO-7. Hardening of the ceramic phase by immersion in water at 100° C., 1 atm, for 55 min.

### Example 2.3

3D-printed scaffolds containing binder solution (11% PLLA, 5% Poloxamer (Kolliphor 407), DCM, water), and α-TCP in a L/P=1.11, resulting in 10.88 wt. % polymeric phase with respect to the total weight of the final composition. Hardening of polymeric phase by evaporation of solvent (DCM) at room temperature and pressure. This step was performed in a clean room ISO-7. Hardening of the ceramic phase by immersion in water at 100° C., 1 atm, for 55 min.

### Example 2.4

3D-printed scaffolds containing binder solution (10.5% PLLA, 6% Poloxamer (Kolliphor 407), DCM, water), and α-TCP in a L/P=1.3, resulting in 12.01 wt. % polymeric phase with respect to the total weight of the final composition. Hardening of polymeric phase by evaporation of solvent (DCM) at room temperature and pressure. This step was performed in a clean room ISO-7. Hardening of the ceramic phase by immersion in water at 100° C., 1 atm, for 55 min.

### Example 2.5

3D-printed scaffolds containing binder solution (11% PLLA, 5% Poloxamer (Kolliphor 407), DCM, water), and α-TCP in a L/P=1, resulting in 9.91 wt. % polymeric phase with respect to the total weight of the final composition. Hardening of polymeric phase by evaporation of solvent (DCM) at room temperature and pressure. This step was performed in a clean room ISO-7. Hardening of the ceramic phase by immersion in water at 100° C., 1 atm, for 55 min.

### Example 3: Printable Self-Setting Inks Based on Polymers Soluble in 1,4-Dioxane

### Example 3.1

3D-printed scaffolds containing binder solution (15% PLLA, 1,4-Dioxane), and α-TCP in a L/P=0.7, 0.8, 0.9, resulting in 9.50, 10.71 and 11.89 wt. % polymeric phase, respectively, with respect to the total weight of the final composition. Hardening of polymeric phase by evaporation of solvent (1,4-Dioxane) at room temperature and pressure. This step was performed in a clean room ISO-7. Hardening of the ceramic phase by immersion in water at 100° C., 1 atm, for 55 min.

### Example 3.2

3D-printed scaffolds containing binder solution (20% PLLA, 1,4-Dioxane), and α-TCP in a L/P=0.6, resulting in 10.71 wt. % polymeric phase with respect to the total weight of the final composition. Hardening of polymeric phase by evaporation of solvent (1,4-Dioxane) at room temperature and pressure. This step was performed in a clean room ISO-7. Hardening of the ceramic phase by immersion in water at 100° C., 1 atm, for 55 min.

### Example 3.3

3D-printed scaffolds containing binder solution (20% PLLA, 1,4-Dioxane), and α-TCP in a L/P=0.5, resulting in 9.09 wt. % polymeric phase with respect to the total weight of the final composition. Hardening of polymeric phase by evaporation of solvent (1,4-Dioxane) at room temperature and pressure. This step was performed in a clean room ISO-7. Hardening of the ceramic phase by immersion in water at 100° C., 1 atm, for 55 min. This configuration showed excellent printability, no shrinkage nor warping of the scaffolds were observed.

### Example 3.4

3D-printed scaffolds containing binder solution (20% PLGA, 1,4-Dioxane), and α-TCP in a L/P=0.5, resulting in 9.09 wt. % polymeric phase with respect to the total weight of the final composition. Hardening of polymeric phase by evaporation of solvent (1,4-Dioxane) at room temperature and pressure. This step was performed in a clean room ISO-7. Hardening of the ceramic phase by immersion in water at 100° C., 1 atm, for 55 min. This configuration showed excellent printability, no shrinkage nor warping of the scaffolds were observed.

### Example 3.5

3D-printed scaffolds containing binder solution (20% PLGA, 1,4-Dioxane), and α-TCP in a L/P=0.45, resulting in 8.26 wt. % polymeric phase with respect to the total weight of the final composition. Hardening of polymeric phase by evaporation of solvent (1,4-Dioxane) at room temperature and pressure. This step was performed in a clean room ISO-7. Hardening of the ceramic phase by immersion in water at 100° C., 1 atm, for 55 min. This configuration showed excellent printability, no shrinkage nor warping of the scaffolds were observed.

### Example 3.6

3D-printed scaffolds containing binder solution (30% PLGA, 1,4-Dioxane), and α-TCP in a L/P=1, resulting in 23.08 wt. % polymeric phase with respect to the total weight of the final composition. Hardening of polymeric phase by evaporation of solvent (1,4-Dioxane) at room temperature and pressure. This step was performed in a clean room ISO-7. Hardening of the ceramic phase by immersion in water at 100° C., 1 atm, for 55 min.

### Example 3.7

3D-printed scaffolds containing binder solution (35% PLGA, 1,4-Dioxane), and α-TCP in a L/P=1, resulting in 25.93 wt. % polymeric phase with respect to the total weight of the final composition. Hardening of polymeric phase by evaporation of solvent (1,4-Dioxane) at room temperature and pressure. This step was performed in a clean room ISO-7. Hardening of the ceramic phase by immersion in water at 100° C., 1 atm, for 55 min.

### Example 3.8

3D-printed scaffolds containing binder solution (35% PLGA, 1,4-Dioxane), and α-TCP in a L/P=0.9, resulting in 23.95 wt. % polymeric phase with respect to the total weight of the final composition. Hardening of polymeric phase by evaporation of solvent (1,4-Dioxane) at room temperature and pressure. This step was performed in a clean room ISO-7. Hardening of the ceramic phase by immersion in water at 100° C., 1 atm, for 55 min

### Example 3.9

3D-printed scaffolds containing binder solution (35% PLGA, 1,4-Dioxane), and α-TCP in a L/P=0.7, resulting in 19.68 wt. % polymeric phase with respect to the total weight of the final composition. Hardening of polymeric phase by evaporation of solvent (1,4-Dioxane) at room temperature and pressure. This step was performed in a clean room ISO-7. Hardening of the ceramic phase by immersion in water at 100° C., 1 atm, for 55 min.

### Example 3.10

3D-printed scaffolds containing binder solution (35% PLGA, 1,4-Dioxane), and α-TCP in a L/P=0.5, resulting in 14.89 wt. % polymeric phase with respect to the total weight of the final composition. Hardening of polymeric phase by evaporation of solvent (1,4-Dioxane) at room temperature and pressure. This step was performed in a clean room ISO-7. Hardening of the ceramic phase by immersion in water at 100° C., 1 atm, for 55 min. This configuration showed excellent printability, no shrinkage nor warping of the scaffolds were observed.

### Example 3.11

3D-printed scaffolds containing binder solution (35% PLGA, 1,4-Dioxane), and α-TCP in a L/P=0.45, resulting in 13.61 wt. % polymeric phase with respect to the total weight of the final composition. Hardening of polymeric phase by evaporation of solvent (1,4-Dioxane) at room temperature and pressure. This step was performed in a clean room ISO-7. Hardening of the ceramic phase by immersion in water at 100° C., 1 atm, for 55 min. This configuration showed excellent printability, no shrinkage nor warping of the scaffolds were observed.

### Example 3.12

3D-printed scaffolds containing binder solution (35% PLGA, 1,4-Dioxane), and α-TCP in a L/P=0.4, resulting in 12.28 wt. % polymeric phase with respect to the total weight of the final composition. Hardening of polymeric phase by evaporation of solvent (1,4-Dioxane) at room temperature and pressure. This step was performed in a clean room ISO-7. Hardening of the ceramic phase by immersion in water at 100° C., 1 atm, for 55 min.

### Example 3.13

3D-printed scaffolds containing binder solution (50% PLGA, 1,4-Dioxane), and α-TCP in a L/P=0.6, resulting in 23.08 wt. % polymeric phase with respect to the total weight of the final composition. Hardening of polymeric phase by evaporation of solvent (1,4-Dioxane) at room temperature and pressure. This step was performed in a clean room ISO-7. Hardening of the ceramic phase by immersion in water at 100° C., 1 atm, for 55 min. This configuration showed excellent printability, no shrinkage nor warping of the scaffolds were observed.

### Example 3.14

3D-printed scaffolds containing binder solution (35% PLGA, 1,4-Dioxane), and α-TCP in a L/P=1, resulting in 25.93 wt. % polymeric phase with respect to the total weight of the final composition. Hardening of polymeric phase by evaporation of solvent (1,4-Dioxane) at room temperature and pressure. This step was performed in a clean room ISO-7. FIGS. 2 (a)-(f) demonstrate the flexibility of a 3D-printed scaffold before hardening of the ceramic phase (i.e., without step d) in the manufacturing process), which may consolidate into a rigid scaffold in situ once implanted in the body and in contact with the body fluid.

### Example 3.15

3D-printed scaffolds containing binder solution (30.84% PCL, 12.61% DCM, 56.55% Chloroform), and α-TCP in a L/P=0.5, resulting in 13.36 wt. % polymeric phase with respect to the total weight of the final composition. Hardening of polymeric phase by evaporation of solvent (dichloromethane, chloroform) at room temperature and pressure. This step was performed in a clean room ISO-7. Hardening of the ceramic phase by immersion in water at 37° C., 1 atm, for 7 days. This configuration showed excellent printability, no shrinkage nor warping of the scaffolds were observed. Example 4. Comparison of the effect of polymer/solvent type and charge on the mechanical properties

### Example 4.1. Three-Point Bending of 3D-Printed Scaffolds Containing PLGA, Printed with Different L/P Ratios

Experimental set-up:

3D-printed bars of 50 mm in length, 4 mm of width and 3 mm of height (50×4×3 mm³, coinciding with the printer X, Y and Z axis, respectively), printed with orthogonal pattern, nozzle inner diameter 24 Ga (0.311 mm), layer height (0.23 mm) and strand separation (250 μm).

Determined by monotonic uniaxial compressive loading in the z-direction (i.e., three-point bending) using a hybrid rheometer (TA Instruments, Discovery HR-2, Germany) equipped with a 3-point bending clamp with a span of 40 mm. The test was run until fracture under displacement-control mode at a crosshead speed of 0.5 mm·min⁻¹.

Twelve 3D-printed samples per condition and the samples were tested in wet conditions (immersed in phosphate-buffered saline solution at 37° C. for 12h). Tested according to ASTM C 1161-02c.

Table 4 provides the results from three-point bending of 3D-printed scaffolds from Examples 3.7-3.10, containing different amount of PLGA in the final compositions, and compared to MimetikOss® 3D (i.e., a pure ceramic scaffold as describes in the patent EP3563881A1 “Synthetic bone graft” following Example 5. Patient-specific defect):

TABLE 4
Three-point bending results from testing 3D-printed scaffolds containing different amount of
PLGA (Examples 3.7-3.10), and compared to MimetikOss ® 3D (i.e., a pure ceramic scaffold
as describes in the patent EP3563881A1 “Synthetic bone graft” following Example 5. Patient-
specific defect). Samples were printed with a 24 Ga nozzle and tested according to ASTM C 1161-02c.

| | MimetikOss ®3D | 35PLGA0.5 | 35PLGA0.7 | 35PLGA0.9 | 35PLGA1.0 |
|---|---|---|---|---|---|
| Ultimate flexural | 2.37 ± 0.90^{a} | 3.18 ± 0.39^{a, b} | 4.01 ± 0.32^{b} | 5.46 ± 1.25^{c} | 7.55 ± 1.06^{d} |
| strength [MPa] | | | | | |
| Flexural | 6.97 ± 3.92^{a} | 18.51 ± 2.28^{b} | 37.72 ± 3.41^{c} | 69.13 ± 12.36^{d} | 76.74 ± 5.71^{d} |
| toughness | | | | | |
| [J · m⁻²] | | | | | |
| Flexural modulus | 1.69 ± 0.49^{a, b} | 1.49 ± 0.24^{a} | 1.81 ± 0.30^{a, b} | 2.09 ± 0.14^{b} | 2.43 ± 0.46^{b, c} |
| [GPa] | | | | | |

Different letters (^{a, b, c, d}) indicate statistically significant differences within each row (analyzed through 1-way ANOVA with Post Hoc Tukey HSD test, with significance level α = 0.05).

It was found that the ultimate flexural strength, work of fracture (i.e. flexural toughness) and flexural modulus increases with increasing amount of PLGA in the final scaffold composition, implying that scaffolds containing a greater amount of PLGA (i.e. 3D-printed from inks with higher L/P ratios) shows a greater enhancement in the mechanical performance during three-point bending.

The work of fracture (WOF) (i.e. flexural and compressive toughness) is an important parameter, especially during surgery when the bone graft is fixated to the host tissue, since an enhanced WOF allows for a better constraint of the bone graft without fracturing. Thus, avoiding micromovements of the bone graft once implanted, which favours early-stage osteointegration. Moreover, as bone grafts with enhanced WOF and plastic deformation can absorb more energy before rupture, they also prevent catastrophic scaffold failures caused by microtensions during the full healing period. There was a clear beneficial effect from the added polymer phase on the mechanical properties.

### Example 4.2. Three-Point Bending Tests of Most Promising 3D-Printed Scaffolds Containing PLGA

The 3D-printed scaffolds containing PLGA from Examples 3.10 and 3.13 are compared to MimetikOss® 3D (i.e., a pure ceramic scaffold as describes in the patent EP3563881A1 “Synthetic bone graft” following Example 5. Patient-specific defect).

### Experimental Set-Up:

3D-printed bars of 50 mm in length, 4 mm of width and 3 mm of height (50×4×3 mm³, coinciding with the printer X, Y and Z axis, respectively), printed with orthogonal pattern, nozzle inner diameter Ga (0.260 mm), layer height (0.2 mm) and strand separation (250 μm).

Determined by monotonic uniaxial compressive loading in the z-direction (i.e., three-point bending) using a hybrid rheometer (TA Instruments, Discovery HR-2, Germany) equipped with a 3-point bending clamp with a span of 40 mm. The test was run until fracture under displacement-control mode at a crosshead speed of 0.5 mm·min⁻¹. Twelve 3D-printed samples per condition and the samples were tested in wet conditions (immersed in phosphate-buffered saline solution at 37° C. for 12h).

Tested according to ASTM C 1161-02c. Table 5 below provides the results from three-point bending (tested according to ASTM C 1161-02c):

TABLE 5
Three-point bending results from testing 3D-printed scaffolds
containing different amount of PLGA (Examples 3.10 and
3.13), and compared to MimetikOss ® 3D (i.e.,
a pure ceramic scaffold as describes in the patent EP3563881A1
“Synthetic bone graft” following Example 5. Patient-
specific defect). Samples were printed with a 25 Ga nozzle
and tested according to ASTM C 1161-02c.

| | MimetikOss ®3D | 35PLGA0.5 | 50PLGA0.6 |
|---|---|---|---|
| Ultimate flexural | 1.62 ± 0.46^{a} | 2.72 ± 0.36^{b} | 3.62 ± 0.45^{c} |
| strength [MPa] | | | |
| Flexural | 5.89 ± 1.74^{a} | 15.55 ± 2.26^{b} | 22.14 ± 3.30^{c} |
| toughness | | | |
| [J · m⁻²] | | | |
| Flexural modulus | 0.99 ± 0.31^{a} | 1.53 ± 0.36^{b} | 1.61 ± 0.39^{b} |
| [GPa] | | | |

Different letters (^{a, b, c}) indicate statistically significant differences within each row (analyzed through 1-way ANOVA with Post Hoc Tukey HSD test, with significance level α = 0.05).

The ultimate flexural strength and the work of fracture (i.e. flexural toughness) are both greater for the compositions containing PLGA (i.e. 35PLGA0.5 and 50PLGA0.6) compared to MimetikOss® 3D, and are further increased when the PLGA content in the final scaffold is increased (i.e. 50PLGA0.6).

### Example 4.3. Compression Tests of Most Promising 3D-Printed Scaffolds Containing PLGA

The 3D-printed scaffolds containing PLGA from Examples 3.10 and 3.13 are compared to MimetikOss® 3D (i.e., a pure ceramic scaffold as describes in the patent EP3563881A1 “Synthetic bone graft” following Example 5. Patient-specific defect).

Experimental set-up: 3D-printed rectangles of 6 mm in cross section and 9 mm in height (6×6×9 mm³, coinciding with the printer X, Y and Z axis, respectively), printed with orthogonal pattern, nozzle inner diameter 25 Ga (0.260 mm), layer height (0.2 mm) and strand separation (250 μm). Determined by monotonic uniaxial compressive loading in the z-direction (Bionix servo-hydraulic test system, MTS Systems, MN, USA). The test was run until fracture under displacement-control mode at a crosshead speed of 0.5 mm·min⁻¹. Twelve 3D-printed samples per condition and the samples were tested in wet conditions (immersed in phosphate-buffered saline solution at 37° C. for 12h). Tested according to ISO 13175-3. Table 6 provides the results:

TABLE 6
Compression results from testing 3D-printed scaffolds containing different
amount of PLGA (Examples 3.10 and 3.13), and compared to MimetikOss ® 3D
(i.e., a pure ceramic scaffold as describes in the patent EP3563881A1 “Synthetic
bone graft” following Example 5. Patient-specific defect). Samples
were printed with a 25 Ga nozzle and tested according to ISO 13175-3.

| | MimetikOss ®3D | 35PLGA0.5 | 50PLGA0.6 |
|---|---|---|---|
| Compressive | 3.73 ± 0.97^{a} | 13.00 ± 0.42^{b} | 26.76 ± 4.48^{c} |
| strength [MPa] | | | |
| Compressive | 165.85 ± 67.23^{a} | 273.39 ± 30.92^{b} | 151.79 ± 17.01^{a} |
| modulus [MPa] | | | |
| Compressive | 913.66 ± 333.00^{a} | 7312.15 ± 885.34^{b} | 43010.93 ± 7590.92^{c} |
| WOF | | | |
| [J · m⁻²] | | | |

Different letters (^{a, b, c}) indicate statistically significant differences within each row (analyzed through 1-way ANOVA with Post Hoc Tukey HSD test, with significance level α = 0.05).

The compressive strength as well as the compressive work of fracture (i.e., compressive toughness) are greater for the compositions containing PLGA (i.e. 35PLGA0.5 and 50PLGA0.6) compared to MimetikOss® 3D, and is further increased when the PLGA content in the final scaffold is increased (i.e. 50PLGA0.6).

### Example 5. Field Emission—Scanning Electron Microscopy (SEM) of 3D-Printed Composite Scaffolds

Experimental set-up: The microstructure of the 3D-printed samples (printed with orthogonal pattern, nozzle inner diameter 25 Ga (0.260 mm), layer height (0.2 mm) and strand separation (250 μm)) were observed through scanning electron microscopy (Phenom XL Desktop SEM, PhenomWorld) run at 15 kV and acquired with a backscattered electron (BSE) detector. Prior to the acquisition, samples were sputter-coated with a thin electron-conductive carbon layer to prevent electrostatic charges (Emitech 950x carbon evaporator, France).

FIG. 3, and 4 show SEM images of the microstructure of 3D-printed scaffolds (Example 3.4, containing PLGA), revealing the needle-like interlocked CDHA crystals in the consolidated ceramic phase (FIG. 3) and the entangled polymeric filaments in a crack in a 3D-printed strand (FIG. 4. (b)), resulting in enhanced mechanical performance compared to pure low-temperature processed ceramics (FIG. 4. (a)).

### Example 6: X-Ray Powder Diffraction—Determination of the Crystalline Phase

Experimental set-up: Phase composition was assessed by X-ray powder diffraction on the powder obtained for each condition after manually crushing the scaffolds in an agate mortar. The diffractometer (D8 Advance, Bruker) equipped with a Cu Ka X-ray tube was operated at 40 kV and 40 mA. Data were collected in 0.02 steps over the 3h range of 10-80° with a counting time of 3 s per step. Phase quantification was performed using the reference intensity ratio method (EVA, Bruker) comparing diffraction patterns of the crystalline structures of alpha-TCP (ICDD PDF 01-070-0346), CDHA (ICDD PDF 01-086-1201) and beta-TCP (ICDD PDF 01-070-2065). See FIG. 5.

Table 7 below shows the results from x-ray powder diffraction of 3D-printed scaffolds containing PLGA from Examples 3.10 and 3.13 are compared to MimetikOss® 3D (i.e., a pure ceramic scaffold as describes in the patent EP3563881A1 “Synthetic bone graft” following Example 5. Patient-specific defect).

TABLE 7
Crystalline phase composition determined by X-ray powder diffraction
from 3D-printed scaffolds containing different amount of PLGA
(Examples 3.10 and 3.13), and compared to MimetikOss ®
3D (i.e., a pure ceramic scaffold as describes in the patent EP3563881A1
“Synthetic bone graft” following Example 5. Patient-specific
defect). The crystalline phases were quantified by intensity ratio
method (EVA). Samples were printed with a 25 Ga nozzle.

| | MimetikOss ®3D | 35PLGA0.5 | 50PLGA0.6 |
|---|---|---|---|
| α-TCP | 12.2% | 15.3% | 12.4% |
| β-TCP | 11.0% | 20.2% | 20.0% |
| CDHA | 76.7% | 64.4% | 67.5% |

### Example 7: FTIR Spectroscopy Analysis

Experimental set-up: Fourier-transform infrared (FTIR) spectroscopy analysis was performed in a Nicolet 6700 FTIR spectrometer (Thermo Fisher Scientific, USA) using the KBr pellet method using 2 mg sample/300 mg KBr. Spectra were recorded in the 4000 to 400 cm⁻¹ range, at 128 scans accumulation and 2 cm⁻¹ resolution. The characteristic bands in the samples were identified by comparing with the typical attributions found in calcium phosphates [1] and in PLGA [2], [3].

The FTIR spectra of the scaffolds containing PLGA (Examples 3.10 and 3.13) both include the typical attributions of the different PO₄³⁻ vibration modes of (v1˜980, v2˜363, v3˜1082 and v4 ˜515 cm⁻¹) typically present in calcium phosphates [1] and which are also found in the control for the ceramic phase (MimetikOss® 3D). Moreover, the absorption bands of HPO₄²⁻ (˜870 cm⁻¹) and OH⁻ (˜631 and 3570 cm⁻¹) which are characteristic of CDHA [1], were also present in the scaffolds containing PLGA (Examples 3.10 and 3.13) and the control (MimetikOss® 3D), demonstrating the presence of the CDHA phase. However, the ceramic phases α-TCP and β-TCP were hardly distinguished in the FTIR spectra due to the overlapping with the CDHA bands, as these three crystalline ceramic phases belongs to the calcium phosphate family.

In addition, scaffolds containing PLGA (Examples 3.10 and 3.13) also showed C—H stretches (˜3000 and ˜2950 cm⁻¹) and C═O stretch (˜1760 cm⁻¹), which are also present in the FTIR spectra for the pure PLGA samples, confirmed the presence of PLGA in the scaffolds.

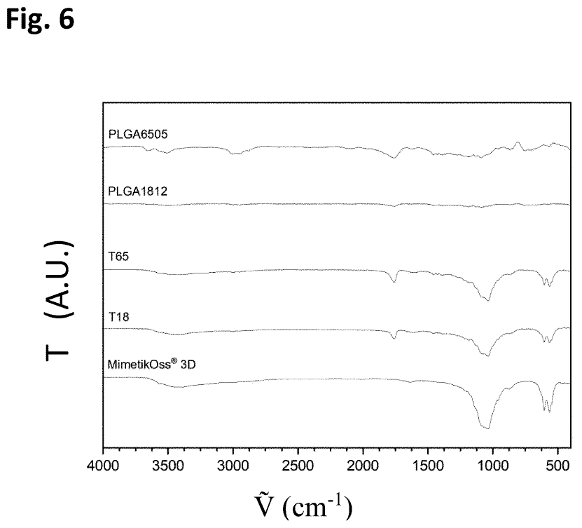

FIG. 6 and Table 8 below shows the results from Fourier-transform infrared (FTIR) spectroscopy analysis of the 3D-printed scaffolds containing PLGA from Examples 3.10 and 3.13, and compared to MimetikOss® 3D (i.e., a pure ceramic scaffold as describes in the patent EP3563881A1 “Synthetic bone graft” following Example 5. Patient-specific defect). Moreover, the FTIR spectra from pure PLGA with different L-lactide:Glycolide molar ratio (82:18 and 65:35) were used as control for the polymeric phase in the 3D-printed scaffolds.

TABLE 8
Fourier-transform infrared (FTIR) spectroscopy analysis from 3D-
printed scaffolds containing different amount of PLGA (Examples
3.10 and 3.13), and compared to MimetikOss ® 3D (i.e.,
a pure ceramic scaffold as describes in the patent EP3563881A1
“Synthetic bone graft” following Example 5. Patient-specific
defect). Samples were printed with a 25 Ga nozzle.

| | Transmittance | Transmittance | Bands | |
|---|---|---|---|---|
| | [a.u.] | [%] | [cm⁻¹] | Groups |
| MimetikOss ®3D | 14.0 | 76.0 | 3650-2800 | St H₂O band |
| | 8.4 | 81.6 | 3570 | OH⁻ band |
| | 5.5 | 84.5 | 1700-1500 | H₂O band |
| | 83.2 | 6.8 | 1040 | PO₄³⁻ band |
| | 36.4 | 53.6 | 962 | PO₄³⁻ band |
| | 17.1 | 72.9 | 876 | HPO₄²⁻ band |
| | 53.0 | 37.0 | 606 | OH⁻ band |
| | 53.3 | 34.7 | 563 | PO₄³⁻ band |
| 35PLGA0.5 | 9.9 | 61.1 | 3650-3100 | St H₂O band |
| | 6.1 | 64.9 | 3570 | OH⁻ band |
| | 4.4 | 66.6 | 3000 | C—H stretches |
| | 4.0 | 67.0 | 2950 | C—H stretches |
| | 17.4 | 53.6 | 1760 | C═O stretch |
| | 6.3 | 64.7 | 1650-1550 | H₂O band |
| | 41.9-19.4 | 29.1-51.6 | 1180-1090 | C—O stretch |
| | 46 | 25 | 1040 | PO₄³⁻ band |
| | 18.3 | 52.7 | 962 | PO₄³⁻ band |
| | 8.5 | 62.5 | 872 | HPO₄²⁻ band |
| | 22.2 | 48.8 | 606 | OH⁻ band |
| | 23.4 | 47.6 | 563 | PO₄³⁻ band |
| | 9.1-8.1 | 61.9-62.9 | 1460-872 | C—H bends |
| 50PLGA0.6 | 7.9 | 78.1 | 3650-3100 | St H₂O band |
| | 5.7 | 80.3 | 3570 | OH⁻ band |
| | 4.3 | 81.7 | 3000 | C—H stretches |
| | 3.8 | 82.2 | 2950 | C—H stretches |
| | 20.9 | 65.1 | 1760 | C═O stretch |
| | 4.3 | 81.7 | 1650-1550 | H₂O band |
| | 45.7-21.5 | 40.3-64.5 | 1180-1090 | C—O stretch |
| | 51.7 | 34.3 | 1040 | PO₄³⁻ band |
| | 18.2 | 67.8 | 960 | PO₄³⁻ band |
| | 7.9 | 78.1 | 872 | HPO₄²⁻ band |
| | 25.3 | 60.70 | 604 | OH⁻ band |
| | 27.3 | 58.7 | 563 | PO₄³⁻ band |
| | 9.1-7.9 | 76.9-78.1 | 1460-872 | C—H bends |
| PLGA8218 | 0.5 | 98.5 | 3650 | O—H stretch |
| | 2.1 | 96.9 | 3500 | O—H end |
| | 1.0 | 98.0 | 3000 | group |
| | 1.1 | 97.9 | 2960 | C—H stretches |
| | 4.0 | 95.0 | 1760 | C—H stretches |
| | 5.6-4.2 | 93.4-94.8 | 1180-1090 | C═O stretch |
| | 2.9-2.2 | 96.1-96.8 | 1460-872 | C—O stretch |
| | | | | C—H bends |
| PLGA6535 | 7.2 | 60.8 | 3650 | O—H stretch |
| | 10.5 | 57.5 | 3500 | O—H end |
| | 12.7 | 55.3 | 3000 | group |
| | 12.4 | 55.6 | 2950 | C—H stretches |
| | 19.1 | 48.9 | 1760 | C—H stretches |
| | 18.6-17.3 | 49.4-50.7 | 1180-1090 | C═O stretch |
| | 14.0-5.6 | 54.0-62.4 | 1460-870 | C—O stretch |
| | | | | C—H bends |

It can be derived that both the formulations 35PLGA0.5 and 50PLGA0.6 (Examples 3.10 and 3.13, respectively) show similar peaks as those found in MimetikOss® 3D and are typical for the CDHA phase. Moreover, stretches related to carbon (i.e. C—H and C═O stretches) were identified in both PLGA compositions, which the pure ceramic scaffold MimetikOss® 3D does not contain. Thus, both the CDHA and PLGA matrixes are present in both PLGA scaffolds, which implies that the CDHA reaction is permitted in the presence of the polymeric phase PLGA. Furthermore, this result corresponds with the x-ray powder diffraction results, which also confirmed the presence of the CDHA crystalline phase in both scaffold compositions containing PLGA.

### BIBLIOGRAPHY

[1]S. Raymond et al., “Accelerated hardening of nanotextured 3D-plotted self-setting calcium phosphate inks,” Acta Biomater., vol. 75, pp. 451-462, July 2018, doi: 10.1016/j.actbio.2018.05.042.
[2]“IR Spectrum Table.” https://www.sigmaaldrich.com/ES/es/technical-documents/technical-article/analytical-chemistry/photometry-and-reflectometry/ir-spectrum-table (accessed Jul. 27, 2022).
[3]C. D'Avila Carvalho Erbetta, “Synthesis and Characterization of Poly(D,L-Lactide-co-Glycolide) Copolymer,” J. Biomater. Nanobiotechnol., vol. 03, no. 02, pp. 208-225, 2012, doi: 10.4236/jbnb.2012.32027.

### Example 8: Apparent Density, Specific Surface Area (SSA) and Helium Pycnometry

Experimental set-up: The apparent density (ρₐₚₚ) of the 3D-printed scaffolds (printed with orthogonal pattern, nozzle inner diameter 25 Ga (0.260 mm), layer height (0.2 mm) and strand separation (250 μm) was calculated as the quotient of the scaffold mass over the scaffold equivalent cubic volume (2×1×1 cm³) obtained from the measurements of the scaffold length, width and height. Prior to measurements the scaffolds were dried at 37° C. overnight (12 h) for complete removal of humidity.

The specific surface area (SSA) of the 3D-printed scaffolds (printed with orthogonal pattern, nozzle inner diameter 25 Ga (0.260 mm), layer height (0.2 mm) and strand separation (250 μm) was determined by nitrogen adsorption using the Brunauer-Emmett-Teller (BET) method (ASAP 2020, Micromeritics, GA, USA). Prior to measurement, samples were degassed in vacuum conditions (10 mmHg) at a holding temperature of 45° C. for 4 h.

The skeletal density of the scaffolds (ρₛₖₑₗ) was assessed by helium pycnometry (AccuPyc 1330, Micromeritics, USA). The powder samples were obtained for each condition by manually crushing the scaffolds in an agate mortar.

The total porosity (Pₜₒₜ) was calculated according to Equation 1 [1]:

$${P}_{t o t} ( \% ) = ( 1 - \frac{{p}_{a p p} [ g . {cm}^{- 1} ]}{{p}_{s k e l} [ g . {cm}^{- 1}} ) * 100 \qquad ( 1 )$$

The nano-microporosity (P_{micro}) was calculated according to Equation 2 [1]:

$${P}_{m i c r o} ( \% ) = ( {V}_{m i c r o} [ {cm}^{3} . {g}^{- 1} ] * {ρ}_{a p p} [ g . {cm}^{- 1} ] ) * 100 \qquad ( 2 )$$

The microporosity P_{macro} was calculated according to Equation 3 [1]:

$${P}_{m a c r o} ( \% ) = {P}_{t o t} ( \% ) - {P}_{m i c r o} ( \% ) \qquad ( 3 )$$

Table 9 shows the specific surface area (SSA), apparent density, skeletal density, total porosity, microposority and maroporosity results of 3D-printed scaffolds containing PLGA from Examples 3.10 and 3.13 and are compared to MimetikOss® 3D (i.e., a pure ceramic scaffold as describes in the patent EP3563881A1 “Synthetic bone graft” following Example 5. Patient-specific defect).

TABLE 9
Specific surface area (SSA), apparent density, skeletal density,
total porosity, microporosity and macropososity of 3D-printed
scaffolds containing different amount of PLGA (Examples 3.10
and 3.13), and compared to MimetikOss ® 3D (i.e.,
a pure ceramic scaffold as describes in the patent EP3563881A1
“Synthetic bone graft” following Example 5. Patient-
specific defect). Samples were printed with a 25 Ga nozzle.

| | MimetikOss ®3D | 35PLGA0.5 | 50PLGA0.6 |
|---|---|---|---|
| SSA | 22.39 | 7.28 | 2.36 |
| [m2 · g⁻¹] | | | |
| Apparent density | 0.73 ± 0.06^{a} | 0.89 ± 0.03^{b} | 1.06 ± 0.05^{c} |
| [g · cm⁻¹] | | | |
| Skeletal density | 2.99 | 2.49 | 2.33 |
| [g · cm⁻¹] | | | |
| Total porosity [%] | 75.59 | 64.26 | 54.51 |
| Microporosity | 27.95 | 12.43 | 0.26 |
| (<10 μm) [%] | | | |
| Macroporosity | 47.64 | 51.82 | 54.25 |
| (>10 μm) [%] | | | |

Different letters (^{a, b, c}) indicate statistically significant differences within each row (analyzed through 1-way ANOVA with Post Hoc Tukey HSD test, with significance level α = 0.05).

### BIBLIOGRAPHY

[1]S. Raymond et al., “Accelerated hardening of nanotextured 3D-plotted self-setting calcium phosphate inks,” Acta Biomater., vol. 75, pp. 451-462, July 2018, doi: 10.1016/j.actbio.2018.05.042.

### Example 9. Mercury Intrusion Porosimetry (MIP)

Experimental set-up: The open porosity and pore entrance size distribution of the 3D-printed scaffolds (printed with orthogonal pattern, nozzle inner diameter 25 Ga (0.260 mm), layer height (0.2 mm) and strand separation (250 μm) were analysed in the range between 0.006 and 360 μm by mercury intrusion porosimetry (MIP, AutoPore IV Micromeritics).

The volume of nano-micropores normalised per unit of mass (V_{micro}) was measured from the sum of the incremental mercury intrusion in the pores smaller than 10 μm.

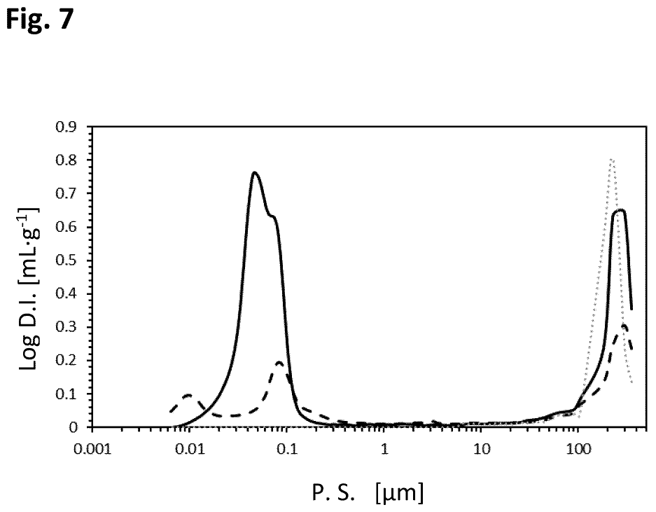

FIG. 7 shows the distribution of pore size for the two tested scaffolds of the invention and the comparative one.

Table 10 below show the macro- micro- and nano pore entrance size of 3D-printed scaffolds (printed with orthogonal pattern, nozzle inner diameter 25 Ga (0.260 mm), layer height (0.2 mm) and strand separation (250 μm)) containing PLGA from Examples 3.10 and 3.13 and are compared to MimetikOss® 3D (i.e., a pure ceramic scaffold as describes in the patent EP3563881A1 “Synthetic bone graft” following Example 5. Patient-specific defect).

TABLE 10
Shows results of pore size entrance analyzed in the range between
0.006 and 360 μm by mercury intrusion porosimetry (MIP)
on 3D-printed scaffolds containing different amount of PLGA
(Examples 3.10 and 3.13), and compared to MimetikOss ® 3D
(i.e., a pure ceramic scaffold as describes in the patent EP3563881A1
“Synthetic bone graft” following Example 5. Patient-
specific defect). Samples were printed with a 25 Ga nozzle.

| Pore entrance | MimetikOss ® | | |
|---|---|---|---|
| size [μm] | 3D | 35PLGA0.5 | 50PLGA0.6 |
| Macroporosity | 230 | 230 | 230 |
| Nano- | 0.05 | 0.01 and 0.08 | — |
| micropososity | | | |

A bimodal pore entrance size distribution can be observed for MimetikOss® 3D, and a trimodal pore entrace size distribution for 35PLGA0.5. Where the pores larger than 10 am corresponded to the macroporosity between 3D-printed strands, and the entrance pore size distribution below 10 μm is related to the nano-microporosity within the strands, which is inherent for each composition. All the scaffolds used for the MIP were 3D-printed following a rectilinear pattern, which made the entrance macropore size distribution overlap in all groups (MimetikOss® 3D, 35PLGA0.5 and 50PLGA0.6). The smallest micropores were observed for the test group 35PLGA0.5, with pore entrance sizes at 0.01 μm, followed by MimetikOss® 3D with pore entrance sizes at 0.05 μm. However, in the composition 50PLGA0.6 only macroporosity was detected by MIP.

### Example 10: Comparison of the Effect of Polymer/Solvent Type and Charge on the Scaffold Biocompatibility

### Example 10.1. Presto Blue Cell-Viability

Osteoblast-like osteosarcoma cells (MG-63, ATCC, USA) were cultured in Dulbecco's Modified Eagle Medium (DMEM) (Gibco, USA) supplemented with 10% foetal bovine serum (FBS), 1% L-glutamine (2 mM) and 1% penicillin/streptomycin (50 U mL⁻¹ and 50 μg mL⁻¹, respectively), all from Gibco, USA. Cells were expanded and maintained at 37° C., 95% humidity and 5% CO₂, and detached using trypsin (Trypsin-EDTA, 0.25 wt./vol. %), phenol red (Gibco, cat. no. 25200056) and centrifuged for 5 min. Cell-viability with MG-63 cell-line at 3 days and 7 days. Evaluation by Presto Blue fluorescence in a micro-plate reader (SYNERGY|HTX, multi-mode reader, BioTek) with a microplate reader and imager software (Gen5, version 3.08, 2006-2009 BioTek Instruments). Incubation for 1h in 37° C., 5% CO2, λex.=560 nm, λem.=590 nm, 100 μl of sample in a 96 wellplate, executed according to providers protocol for Presto Blue). MimetikOss® 3D (i.e., a pure ceramic scaffold as describes in the patent EP3563881A1 “Synthetic bone graft” following Example 5. Patient-specific defect, consisting of 70 wt. % CDHA and 30 wt. % β-TCP) and is used as control, 50PLGA0.6 is a composite scaffold (Example 3.13: 50 wt. % PLGA/1,4-Dioxane, L/P=0.6), 35PLGA0.5 is a composite scaffold (Example 3.10: 35 wt. % PLGA/1,4-Dioxane, L/P=0.5).

Experimental set-up: 3D-printed discs of 2 mm in height and 6 mm in diameter (2×6 mm³), printed with orthogonal pattern, nozzle inner diameter 25 Ga (0.260 mm), layer height (0.2 mm) and strand separation (250 μm).

Cell response was studied in static 3D culture conditions in 3D-printed samples. Scaffolds (discs of 6 mm in diameter and 2 mm in height) were placed in 24-well cell culture plates. Samples were then preconditioned for 24 h with 2 mL of Dulbecco's Modified Eagle Medium supplemented with 10% fetal bovine serum, 2 mM L-glutamine, 50 mL penicilli and 50 mg mL streptomycin. Afterwards, the medium was removed and 0.6 M cells (MG-63 cancerous cell-line in passages 96 to 102) were seeded on each scaffold in a 100 μl medium drop. MG-63 were allowed to attach during 30 min at 37° C. and 5% CO₂ and then 2 mL of fresh medium was gently added. After 1 day of static cell culture, the scaffolds were moved to a new well plate. Fresh medium was changed each 24 h.

Immunofluorescent staining was used to visualise cell morphology and for cell counting. After 7 days of culture, the scaffolds were rinsed in PBS-glycine and cells were fixed for 20 min in 4% paraformaldehyde solution. Cells were permeabilised for 15 min with Triton X-100 (0.05%) and blocked for 30 min in PBS-bovine serum albumin (BSA; 1%). Actin filaments were stained with TRITC-conjugated phalloidin (Sigma-Aldrich, USA) and nuclei were counterstained with DAPI (40,6-diamidino-2-phenylindole, 1 lg/ml). Images were acquired with a fluorescence confocal laser scanning microscopy microscope (Carl ZEISS, LSM 800) and processed with a confocal image processing software (ZEN 2.3, Bule edition). Experiments were performed in triplicates for statistical analysis.

Cell-viability results from the 3D-printed scaffolds demonstrate excellent biological properties in vitro (Table 11 and Table), where the scaffolds containing PLGA show equal (i.e. 35PLGA0.5) or enhanced (i.e. 50PLGA0.6) biological performance compared to the pristine MimetikOss® 3D (i.e., a pure ceramic scaffold as describes in the patent EP3563881A1 “Synthetic bone graft” following Example 5. Patient-specific defect).

TABLE 11
Cell-viability results measured by Presto Blue. MG-63 cell-line
was seeded on 3D-printed scaffolds containing different amount
of PLGA (Examples 3.10 and 3.13), and compared to MimetikOss ® 3D
(i.e., a pure ceramic scaffold as describes in the patent EP3563881A1
“Synthetic bone graft” following Example 5. Patient-specific
defect). Samples were printed with a 25 Ga nozzle and tested according
to the manufacturers protocol. Cell-viability results are normalized
with values obtained from MimetikOss ® 3D at 3 days.

| MG-63 [%] | MimetikOss ®3D | 35PLGA0.5 | 50PLGA0.6 |
|---|---|---|---|
| 7 days | 188.29 ± 26.12^{c} | 176.67 ± 25.10^{c} | 227.52 ± 26.69^{d} |

Different letters (^{a, b, c, d}) indicate statistically significant differences (analyzed through 1-way ANOVA with Post Hoc Tukey HSD test, with significance level α = 0.05).

TABLE 12
Cell counting results. MG-63 cell-line was seeded on 3D-printed
scaffolds containing different amount of PLGA (Examples 3.10
and 3.13), and compared to MimetikOss ® 3D (i.e.,
a pure ceramic scaffold as describes in the patent EP3563881A1
“Synthetic bone graft” following Example 5. Patient-
specific defect). Samples were printed with a 25 Ga nozzlel.

| MG-63 | | | |
|---|---|---|---|
| [no. cells · μm⁻²] | MimetikOss ®3D | 35PLGA0.5 | 50PLGA0.6 |
| 1 day | 1271 ± 442^{a} | 810 ± 507^{a} | 1479 ± 890^{a} |
| 7 days | 2983 ± 195^{a} | 3787 ± 50^{b} | 4042 ± 183^{c} |

Different letters (^{a, b, c}) indicate statistically significant differences within each row (analyzed through 1-way ANOVA with Post Hoc Tukey HSD test, with significance level α = 0.05).

### Example 10.2. Ion-Exchange Evaluation by Inductively Coupled Plasma Mass Spectrometry (ICP-MS)

ICP-MS analysis for calcium and phosphor ion-release, from different 3D-printed scaffolds to 2 mL complete culture media, for a total of 7 days. MimetikOss® 3D (i.e., a pure ceramic scaffold as describes in the patent EP3563881A1 “Synthetic bone graft” following Example 5. Patient-specific defect, consisting of 70 wt. % CDHA and 30 wt. % β-TCP), T65 is a composite scaffold (Example 3.13: 50 wt. % PLGA/1,4-Dioxane, L/P=0.6), T18 is a composite scaffold (Example 3.10: 35 wt. % PLGA/1,4-Dioxane, L/P=0.5).

Experimental set-up: 3D-printed discs of 2 mm in height and 6 mm in diameter (2×6 mm³), printed with orthogonal pattern, nozzle inner diameter 25 Ga (0.260 mm), layer height (0.2 mm) and strand separation (250 μm).

ICP-MS measurements revealed that the ion exchange is greater for MimetikOss® 3D than for the compositions containing PLGA, especially concerning calcium ions (Tables 13 and 14). Moreover, the PLGA-containing scaffolds show globally a more stable ion-concentrations in the culture media over time compared to MimetikOss® 3D, which indicates that the uptake and release of both calcium and phosphor is lower than the control. Thus, may favorize cell-adhesion and proliferation on these scaffolds.

TABLE 13
ICP results for evaluation of the calcium ion exchange from
3D-printed scaffolds containing different amount of PLGA (Examples
3.10 and 3.13), and compared to MimetikOss ® 3D (i.e.,
a pure ceramic scaffold as describes in the patent EP3563881A1
“Synthetic bone graft” following Example 5. Patient-
specific defect). Samples were printed with a 25 Ga nozzle.

| Ca²⁺ | | | |
|---|---|---|---|
| (mM) | MimetikOss ®3D | 35PLGA0.5 | 50PLGA0.6 |
| 1 day | 0.68 ± 0.06^{a} | 1.42 ± 0.12^{b} | 1.79 ± 0.08^{c} |
| 2 days | 1.17 ± 0.08^{a} | 1.68 ± 0.03^{b} | 1.78 ± 0.05^{c} |
| 3 days | 1.26 ± 0.07^{a} | 1.69 ± 0.01^{b} | 1.74 ± 0.02^{b} |
| 4 days | 1.39 ± 0.07^{a} | 1.77 ± 0.01^{b} | 1.75 ± 0.06^{b} |
| 8 days | 1.58 ± 0.06^{a} | 1.83 ± 0.01^{b} | 1.84 ± 0.03^{b} |

Different letters (^{a, b, c}) indicate statistically significant differences within each row (analyzed through 1-way ANOVA with Post Hoc Tukey HSD test, with significance level α = 0.05).

TABLE 14
ICP results for evaluation of the phosphorous ion exchange from
3D-printed scaffolds containing different amount of PLGA (Examples
3.10 and 3.13), and compared to MimetikOss ® 3D (i.e.,
a pure ceramic scaffold as describes in the patent EP3563881A1
“Synthetic bone graft” following Example 5. Patient-
specific defect). Samples were printed with a 25 Ga nozzle.

| P | | | |
|---|---|---|---|
| (mM) | MimetikOss ®3D | 35PLGA0.5 | 50PLGA0.6 |
| 1 day | 1.22 ± 0.13^{a} | 1.09 ± 0.04^{b} | 1.23 ± 0.02^{a} |
| 2 days | 0.94 ± 0.02^{a} | 1.09 ± 0.02^{b} | 1.15 ± 0.03^{c} |
| 3 days | 0.95 ± 0.01^{a} | 1.12 ± 0.01^{b} | 1.14 ± 0.02^{b} |
| 4 days | 0.96 ± 0.05^{a} | 1.15 ± 0.01^{b} | 1.14 ± 0.04^{b} |
| 8 days | 1.00 ± 0.04^{a} | 1.14 ± 0.01^{b} | 1.15 ± 0.02^{b} |

Different letters (^{a, b, c}) indicate statistically significant differences within each row (analyzed through 1-way ANOVA with Post Hoc Tukey HSD test, with significance level α = 0.05).

### Example 11: Further Mechanical Tests

### Scaffolds Preparation

The ceramic control scaffolds (CTRL) were obtained from an extrudable ink, composed of a mixture of a poloxamer 407 (BASF, Germany) hydrogel with a polymer concentration of 30 wt./vol. % and α-TCP powder, with a liquid to powder (L/P) ratio of 0.45, which formed a ceramic suspension with adequate pseudo-plastic behaviour, and was prepared as described in the patent EP3563881, following Example 5. The scaffolds printed with PLGA (PLGA-1) were prepared from an extrudable ink based on an organogel binder. First, the organogel was prepared by dissolving medical grade PLGA as received (Corbion, The Netherlands) in liquid 1,4-Dioxane (Fisher scientific, USA) with a 35 wt./wt. % polymer concentration. The two compounds were mixed in a dual asymmetric centrifugal mixer (DAC 150, Speedmixer, USA) at 3500 rpm for 30 minutes to achieve complete dissolution of the PLGA, resulting in an homogeneous and transparent organogel. Thereafter, α-TCP powder was added to the PLGA organogel at a liquid to powder ratio of 0.5 wt./wt. and mixed in a dual asymmetric centrifugal mixer (DAC 150, Speedmixer, USA) at 2100 rpm for 4 minutes to ensure homogeneous distribution of the ceramic particles in the ink. After mixing, both hydrogel- and organogel-based inks were immediately introduced in a 5 mL cartridge (QuantX™, Syringe Barrel, Fisnar, USA) and extruded through a ø260 μm nozzle (Smooth Flow Tapered Dispensing Tip, Gauge 25, Fisnar, USA) by a costume-made direct ink writer (DIW) 3D printer (Heavy Duty Paste Extruder, CIM-UPC, Spain) at ambient temperature (18-25° C.).

All scaffolds were printed according to an orthogonal pattern with a 0-90° lay-down, with a strand-to-strand separation of 260 μm, a layer height of 200 μm, which corresponds to a layer overlap of 25% in the Z-direction, at a deposition speed of 10 mm/s. Different geometries were printed: blocks (1×1×2 cm³), bars (3×4×50 mm³), rectangular prisms (6×6×9 mm³) and discs (6 mm ø×2 mm). These 3D constructs were designed with the software Meshmixer (Meshmixer 3.5, 2018, Autodesk, Inc.) and thereafter exported as stereolithography (STL) files. These printing parameters together with the STL files were converted into a numerical control programming language (G-code) with the software Simplify 3D (V. 3.0.2, 2015, SIMPLIFY3D®) before printing.

Once finished printing, the 3D-printed green CTRL constructs were immediately submitted to a vapour treatment (10 min, 100° C.) (S. Raymond et al., “Accelerated hardening of nanotextured 3D-plotted self-setting calcium phosphate inks,” Acta Biomater., vol. 75, pp. 451-462, July 2018, doi: 10.1016/j.actbio.2018.05.042) to obtain minimal cohesion of the samples for manipulation before the hydrothermal consolidation process in immersed conditions. On the other hand, the same cohesion before immersion of the samples was achieved for the PLGA-I samples by leaving the constructs 5 minutes at room temperature for solvent evaporation and rigidification of the polymeric phase. Subsequently, both conditions were subjected to a hydrothermal immersed consolidation process at 121° C. in an autoclave. Briefly, α-TCP hydrolyses through an accelerated reaction into CDHA, which results in the hardening of the ceramic phase in the scaffolds.

The PLGA-coated ceramic scaffolds (PLGA-C) were prepared using the 3D-printed and consolidated ceramic scaffolds described above (CTRL), and a PLGA solution. First, PLGA was dissolved in 1,4-Dioxane using the same process as described above, however, with a 10 wt./vol. % polymer concentration. Once the PLGA was completely dissolved, the 3D-printed pure ceramic scaffolds (i.e. finished CTRL scaffolds) were immersed in the homogenous PLGA-solution. The scaffolds were infiltrated by the PLGA solution under vacuum for 4 h at room temperature (25° C.). After infiltration, the scaffolds were removed from the PLGA solution, excessive solution was wiped off and the coated scaffolds were left to dry at room temperature for 3 min. Finally, the coated scaffolds were rinsed in distilled water by submersion in a 900 mL beaker for 30 s x 1 to remove the solvent, resulting in a 3D-printed ceramic scaffold coated with a thin PLGA layer.

All samples were sterilised by wet heat in an autoclave (15 min, 121° C.) before further characterisation.

### Cell Culture

Human bone marrow-derived mesenchymal stem cells (hMSC, #1319B, ATCC, USA) were cultured in advanced Dulbecco's Modified Eagle Medium (AdvDMEM) (Gibco, USA) supplemented with 10% foetal bovine serum (FBS), 1% L-glutamine (2 mM) and 1% penicillin/streptomycin (50 U mL⁻¹ and 50 μg mL⁻¹, respectively), all from Gibco, USA. Cells were expanded and maintained at 37° C., 95% humidity and 5% CO2. Cell response was studied in static 3D culture conditions in direct contact with the 3D-printed samples (discs of 6 mm diameter and 2 mm height). Prior to cell seeding, scaffolds of all conditions were placed in a 24-well cell culture plate and preconditioned for 24 h in 2 mL of supplemented AdvDMEM. Subsequently, the medium was removed and 0.2 M cells (hMSC, passage 5) concentrated in a 60 μL supplemented AdvDMEM drop were seeded on top of each scaffold and incubated for 30 min to allow cells for attachment before 2 mL of supplemented AdvDMEM was added to each sample. All scaffolds with attached cells were transferred to a new 24-well cell culture plate 24 h after seeding and 2 mL of fresh medium was added gently to each sample. The samples were incubated for 1 day, 7 days and 21 days. The medium was changed for fresh supplemented AdvDMEM every 24 h.

### Cell Morphology and Counting

Samples from each time point were rinsed with phosphate buffered saline (PBS) (Dulbecco, Biowest SAS, France). Subsequently, hMSC cells were fixed with 4% paraformaldehyde in PBS (PFA, Sigma-Aldrich, USA) for 30 min at room temperature, thereafter permeabilised with 0.1% Tween20 in PBS (Sigma-Aldrich, USA) for 30 min and finally blocked using SuperBlock™ (TBS) (ThermoScientific, Ref. 37535, USA) for 2 h. Successively, actin filaments (F-Actin) were stained with 1:300 PBS dilution of Alexa Fluor™ 546 Phalloidin (Invitrogen™, USA) during 1 h to allow staining of the cytoskeleton and the nuclei were stained with 1:1000 PBS dilution of 4′,6-diamidino-2-phenylindole (DAPI, LifeTechnologies, USA) for 10 min. Representative fluorescence images were acquired by a confocal laser scanning microscope (CLSM) (LSM 800, Zeiss, Germany), equipped with four different objectives: 5× air objective, 10× air objective, 20× water objective and 40× oil objective and operated with ZEN 2.3 software (Zeiss, Germany), using 493 nm and 557 nm as excitation wavelengths and 400-555 nm and 540-700 nm as detection wavelengths for DAPI and Phalloidin detection, respectively. Subsequently, the nuclei on the upper surface-area of the second strand layer were counted in randomised images from each scaffold condition (3 areas per image and 3 images per condition) using an image analysis software (ImageJ2, NIH, MD, USA). Additionally, the cell area was calculated from randomised images (3 images per condition) using the CLSM provider software (ZEN 2.3, Zeiss, Germany) and normalised relative the number of nuclei.

### SEM Images

The microstructure of the 3D-printed samples was observed through scanning electron microscopy (FIB/SEM, Neon 40, Zeiss, Germany) run at 5 kV and acquired with a through-the-lens backscattered electron (BSE) detector. Prior to the acquisition, samples were sputter-coated with a thin electron-conductive carbon layer (Emitech K950X carbon evaporator, France).

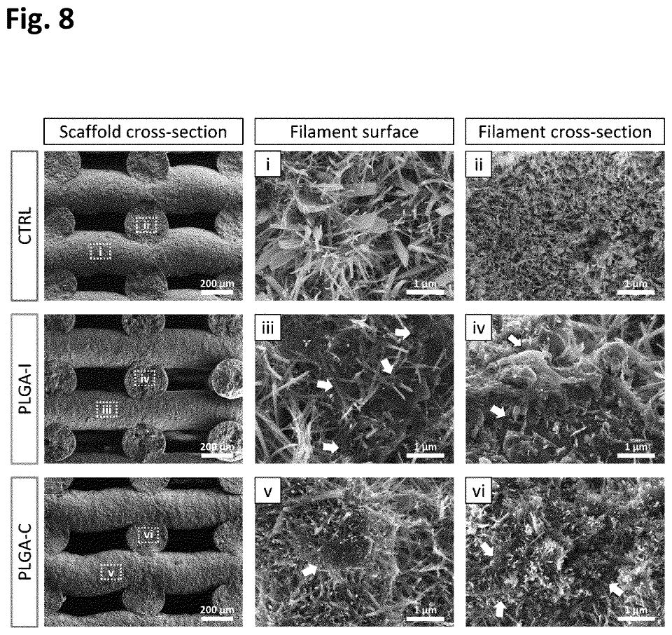

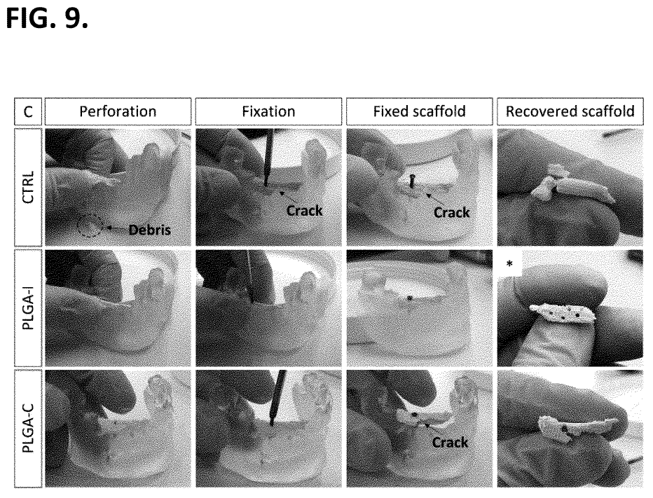

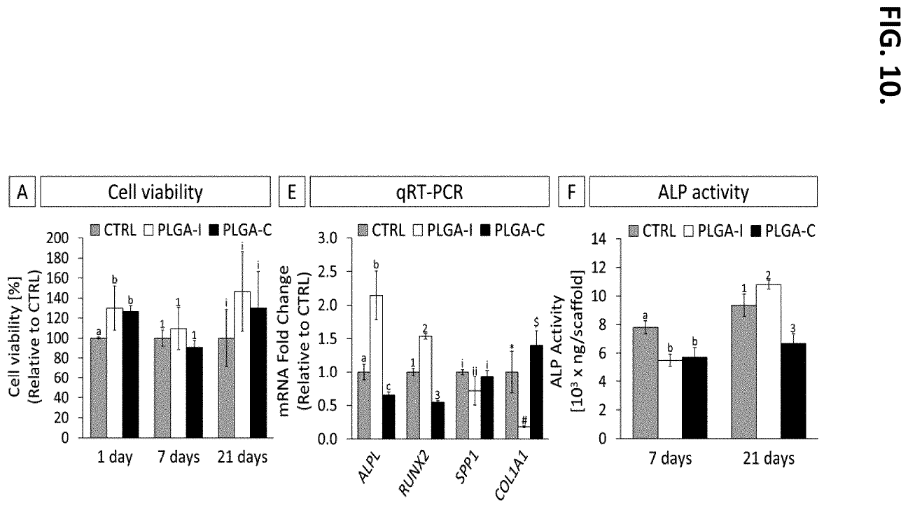

The scanning electron microscopy (SEM) images revealed a microstructure of needle-like nanocrystals (FIG. 8) in all three groups, typical for the CDHA crystalline phase set in hydrothermal conditions. Additionally, the polymeric phase (PLGA) was observed on the filament surface and the filament cross-section, entangled with the CDHA needle-like crystals, in both composite scaffolds (PLGA-I and PLGA-C, indicated by white arrows). Moreover, the presence of the polymeric phase was more evidenced in the cross-section of the filaments in PLGA-I, and conversely on the filament surface in PLGA-C. The different preferential positioning of the polymeric phase in the two composite configurations was predictable: favourable in the filament cross-section in the PLGA-I, with the hydrophobic PLGA merging inside the filament, protected from the water of the setting treatment by the hydrophilic CDHA forming crystals during the setting reaction of the ink. Conversely, in the PLGA-C, the polymeric phase was preferentially situated on the filament surface rather than in the core, with the already set CDHA structure impeding the viscous polymer solution from penetrating inside the filaments during the coating step. Comparing the microstructure in the two composites with the control samples, it is noticeable that the incorporated PLGA phase conceals a part of the microporosity.

### Fixability Test

To verify the mechanical properties in fixability of the scaffolds, a screwability test of the three different scaffold types was performed. For this purpose, a real scaffold design to face a vertical and horizontal reconstruction of a knife-edge ridge in the jaw was used as a model. The defect reconstruction was designed from real patient CBCT images by segmenting the bone area around the indication (Mimics Innovation Suite, Medical, V.25, Materialise) and exported as a STL file. Based on the previous segmented anatomical model from the patient, the bone graft was designed with the software Meshmixer (Meshmixer 3.5, 2018, Autodesk, Inc.) and exported as STL file.

CTRL and PLGA-I scaffolds were 3D-printed with orthogonal pattern with a 0-90° lay-down, a nozzle inner diameter of 25 Ga (260 μm 0), a layer height of 200 μm and with a strand-to-strand separation of 260 μm. These printing parameters together with the STL file were converted into a numerical control programming language (G-code) with the software Simplify 3D (V. 3.0.2, 2015, SIMPLIFY3D®) before printing. An anatomical biomodel with the corresponding bone defect obtained from the previous bone segmentation was 3D-printed in transparent photopolymer resin (3D UV resin, WEISTEK, China) by a digital light processing 3D printer (Mars 3, ELEGOO, Inc., China). Subsequently, scaffolds (CTRL, PLGA-I, PLGA-C) were perforated with a 1.2 mm diameter drill (smartDrive®, ref. 26-975-45-71, KLS Martin, Germany) mounted on a dental straight nose surgical handpiece (Seasky, China) connected to a micromotor (Marathon-Ill, SHIYANY, China) and fixated on the anatomical biomodel with a 1.5 mm diameter and 7 mm in length dental screw (maxDrive®, ref. 25-875-07-09, KLS Martin, Germany).

As demonstrated in FIG. 8, the bioceramic scaffolds (CTRL) could be perforated with a ø1.2 mm drill, however, the scaffold broke in three pieces during the fixation of a ø1.5 mm dental screw. Thus, could not be completely fixated to the anatomical biomodel. The PLGA-C was successfully perforated without signs of fracture, however, a crack formed and propagated during the anchoring of the screw. Conversely to the CTRL, the PLGA-C continued to further open the crack during the fixation process and, instead of breaking in several pieces, resisted catastrophic failure and stayed as one piece. The PLGA-I could be both successfully perforated and fixated to the anatomical biomodel while staying intact and without signs of fracture. Additionally, during the perforation step, the CTRL samples generated significantly more debris compared to the PLGA-I and PLGA-C samples (indicated by black arrow in FIG. 8), suggesting that the composite scaffolds held greater structural integrity when submitted to drilling, in comparison with the CTRL. After performing the fixation test, the surviving scaffolds were further perforated to assess its resistance to close adjacent drill holes. The PLGA-I condition (i.e. the only surviving scaffold) could endure at least 5 perforated holes (ø1.2 mm) with adjacent holes separations of down to 1 mm without breaking, while the bioceramic scaffold only tolerated to be perforated once. Hence, the enhanced mechanical properties, supplemented by the added polymeric phase in the composite scaffolds, improved the screwability and fixability of the bone grafts, which is a beneficial advancement towards their clinical application as personalised bone grafts.

Influence of the PLGA Phase on the Biological Performance on hMSC Cell Line and Osteodifferentiation

First, to ensure that the cell seeding was homogenous and that hMSC could proliferate on the 3D structures, cells were seeded on CTRL samples and incubated for 1 and 7 days for fluorescence imaging of the complete scaffolds. Fluorescence staining revealed that hMSC were present in the entire scaffold structure at both timepoints, evidencing a homogeneous seeding and proliferation of the cells in the samples (FIG. S2—Supplementary). This is in accordance with the images of MG-63 cell line from previous section. Important to note that the cell proliferation and growth of hMSC is slower and require longer time compared to MG-63. Thus, it was expected to observe a lower cell number of hMSC in the scaffolds compared to MG-63 at 7 days and longer cell culture times (21 days) were required.

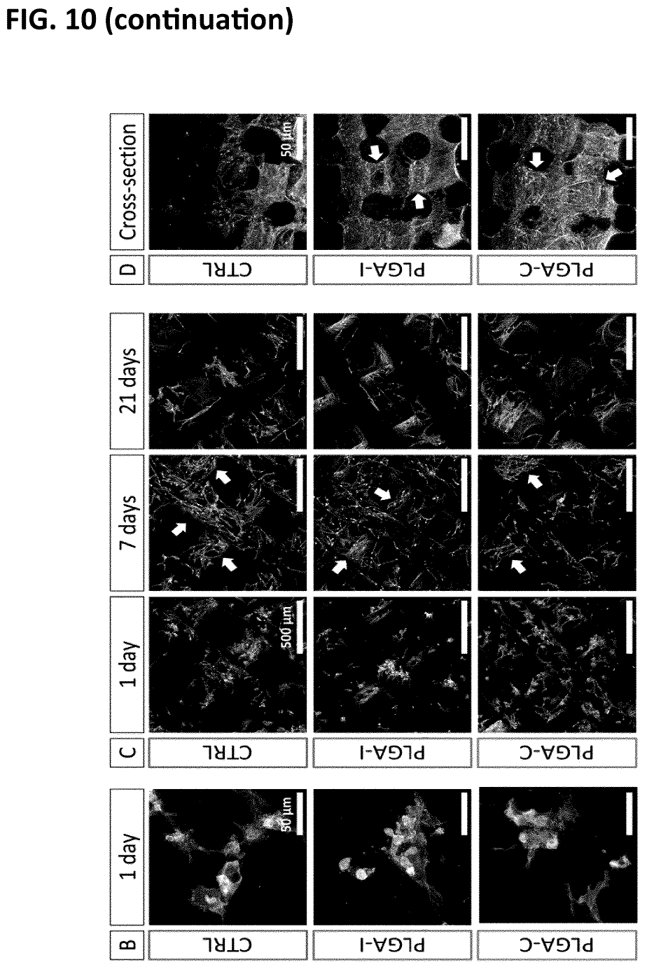

Cell viability at 1 day showed improved biocompatibility from the PLGA containing scaffolds compared to the CTRL samples, but no significant differences were observed between PLGA-I (130±22%) and PLGA-C(127±6%). At 7 days and 21 days the cell viability for the PLGA-containing scaffolds did not present statistically significant differences with the CTRL samples, with values of 110±22% and 91±6% at day 7 and 146±40% and 130±36% at day 21 for the PLGA-I and PLGA-C, respectively (FIG. 10A).

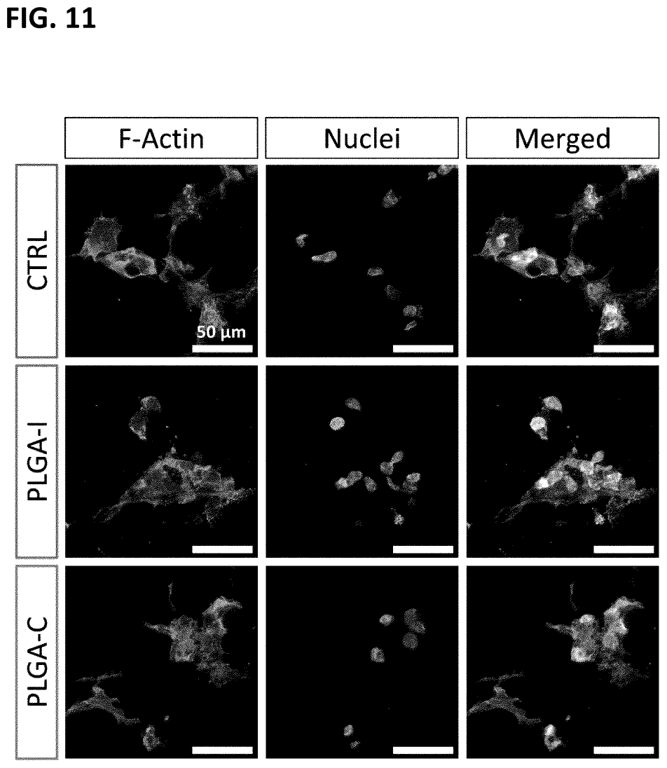

The cell morphology, cell-adhesion and proliferation were studied by fluorescence images acquired by a confocal laser scanning microscope (CLSM) (LSM 800, Zeiss, Germany)), equipped with four different objectives: 5× air objective, 10× air objective, 20× water objective and 40× oil objective and operated with ZEN 2.3 software (Zeiss, Germany), using 493 nm and 557 nm as excitation wavelengths and 400-555 nm and 540-700 nm as detection wavelengths for DAPI and Phalloidin detection, respectively. At 1 day, hMSC were able to attach to the filaments in all three test conditions and their cytoskeleton were already starting to spread (FIG. 10B, separated images of the F-Actin, nuclei and merged images for the three conditions at 1 day are shown in FIG. 11 in supplementary information). At 7 days, cells had proliferated and showed a network of more interconnected cytoskeletons, bridging between cells (indicated by white arrows in FIG. 10C). Finally, at 21 days the cells had continued to proliferate and started to cover the surface of the scaffold filaments (FIG. 10C). The lattice was more appreciated and exhibited from the confocal images taken from the scaffolds cross-section (FIG. 10D) instead of the z-direction, where an additional gradient in the cell growth was also revealed, especially in the CTRL samples. These images show that, while only the bottom layer strands were completely covered by cells at 21 days in the CTRL scaffolds, both the PLGA containing scaffolds promoted cell growth in all filament layers, where all strands from bottom to top were completely covered by cells, revealing that the cells could migrate through the full composite scaffolds. Moreover, it was also observed that the cells started to bridge the macropores between strands in the PLGA-I and PLGA-C conditions (indicated by white arrows in FIG. 10D), however, no penetration within the strands was observed due to the small pore-size entrances of the microporosity (FIG. 10D).

Gene expression of osteogenic marker was evaluated at 21 days in order to determine the osteogenic differentiation of hMSC in direct contact with the developed composite configurations and compared to pristine bioceramic samples.

To that end, gene expression of osteogenic markers was assessed by real-time quantitative reverse transcription polymerase chain reaction (qRT-PCR). Isolation, extraction and purification of RNA from the different samples were performed using a RNAeasy Mini Kit (Qiagen, Germany) following the manufacturer's protocol (n=3). RNA quantification was carried out by spectrophotometry using a Take3 microvolume plate (Bio-Tek, USA). Subsequently, RNA was retrotranscribed to cDNA employing Maxima First Strand cDNA Synthesis Kit for qRT-PCR, with dsDNase (Thermo Scientific, #K1671). The gene expression was assessed by QuantiNova SYBR™ Green PCR Kit (Qiagen, Germany) using a Magnetic Induction Cycler (MIC) qPCR equipment (Bio Molecular Systems, Australia) and its corresponding software (micPCR, v2.12.6, Bio Molecular Systems, Australia). Glyceraldehyde 3-phosphate dehydrogenase (GAPDH) was used as housekeeping gene to normalise mRNA expressions and the relative gene expression levels were evaluated using 2^{ΔΔ-Ct} method. Primer sequences are shown in supplementary Table 15:

TABLE 15
List of DNA sequences of forward and reverse primers used for the qRT-PCR in this study.

| Gene name | Gene ID | Forward primer (5′ → 3′) | Reverse primer (5′ → 3′) |
|---|---|---|---|
| Glyceraldehyde- | GAPDH | TTG CCA TCA ATG ACC CCT TCA | CGC CCC ACT TGA TIT TGG A |
| 3-phosphate | | (SEQ ID NO: 1) | (SEQ ID NO: 2) |
| dehydrogenase | | | |
| Collagen alpha- | COL1A1 | AGGTCCCCCTGGAAAGAA (SEQ ID | AATCCTCGAGCACCCTGA (SEQ |
| 1(I) chain | | NO: 3) | ID NO: 4) |
| Runt-related | RUNX2 | AAATGCCTCCGCTGTTATGAA | GCTCCGGCCCACAAATCT (SEQ |
| transcription | | (SEQ ID NO: 5) | ID NO: 6) |
| factor 2 | | | |
| Alkaline | ALPL | ATCTTTGGTCTGGCTCCCATG (SEQ | TTTCCCGTTCACCGTCCAC |
| phosphatase, | | ID NO: 7) | (SEQ ID NO: 8) |
| tissue- | | | |
| nonspecific | | | |
| isozyme | | | |
| Osteopontin | SPP1 | AGCTGGATGACCAGAGTGCT (SEQ | TGAAATTCATGGCTGTGGAA |
| | | ID NO: 9) | (SEQ ID NO: 10) |

Runt-related transcription factor 2 (RUNX2) is known to be implied in the osteogenic differentiation of MSC, especially at an early stage of the differentiation process, and regulates bone matrix formation (T. Komori, “Whole Aspect of Runx2 Functions in Skeletal Development,” Int. J. Mol. Sci., vol. 23, no. 10, p. 5776, May 2022, doi: 10.3390/ijms23105776).

Collagen alpha-1(I) chain (COL1A1) is usually expressed in an earlier stage of the osteoblast differentiation sequence and is related to the formation of collagen type I in the extracellular matrix before mineralisation (T. Komori, 2022, supra).

Alkaline phosphatase (ALPL) is also known to be involved in the differentiation of MSC into osteoblasts (J. M. Sadowska et al., “In vitro response of mesenchymal stem cells to biomimetic hydroxyapatite substrates: A new strategy to assess the effect of ion exchange,” Acta Biomater., vol. 76, pp. 319-332, August 2018, doi: 10.1016/j.actbio.2018.06.025; T. M. Liu and E. H. Lee, “Transcriptional Regulatory Cascades in Runx2-Dependent Bone Development,” Tissue Eng. Part B Rev., vol. 19, no. 3, pp. 254-263, June 2013, doi: 10.1089/ten.teb.2012.0527). Nonetheless, the ALP enzyme is involved in the mineralization process (S. Sekaran et al., “The Physiological and Pathological Role of Tissue Nonspecific Alkaline Phosphatase beyond Mineralization,” Biomolecules, vol. 11, no. 11, p. 1564, October 2021, doi: 10.3390/biom11111564; H. Orimo, “The Mechanism of Mineralization and the Role of Alkaline Phosphatase in Health and Disease,” J. Nippon Med. Sch., vol. 77, no. 1, pp. 4-12, 2010, doi: 10.1272/jnms.77.4; U. Sharma et al., “Alkaline Phosphatase: An Overview,” Indian J. Clin. Biochem., vol. 29, no. 3, pp. 269-278, July 2014, doi: 10.1007/s12291-013-0408-y), so highly upregulated ALPL expressions are considered a sign for active osteoblasts in the process of mineralization of the bone matrix.

Osteopontin, also known as secreted phosphoprotein 1 (SPP1), is an osteogenic marker involved in the regulation of the mineralisation process (S. Sekaran et al., “The Physiological and Pathological Role of Tissue Nonspecific Alkaline Phosphatase beyond Mineralization,” Biomolecules, vol. 11, no. 11, p. 1564, October 2021) and is often used as a late osteogenic marker in cell differentiation assessments. Gene expression assessments by real-time quantitative reverse transcription polymerase chain reaction (qRT-PCR) showed that the ALPL expression was enhanced by 2-fold in the PLGA-I, compared to the CTRL. Similar tendency was observed for RUNX2 expression, where the PLGA-I increased the gene expression by 1.5-fold compared to the CTRL. Regarding the SPP1 expression, the PLGA containing samples presented equal performance as the CTRL samples, whereas the COL1A1 expression was insignificantly downregulated in the PLGA-I samples compared to the CTRL (FIG. 10D). The upregulation of RUNX2 and ALPL genes with a downregulated COL1A1 expression from the PLGA-I samples, suggested that the hMCS presented higher differentiation into mature osteoblasts in the process of mineralisation compared to the CTRL samples.

To verify that the ALPL gene expression was translated into functional protein expression, the ALP activity at 7 and 21 days was assessed. Both types of PLGA samples showed a decrease of ALP activity compared to CTRL samples at day 7 (FIG. 10F). Nevertheless, the results revealed an increasing trend of the ALP activity with culture time in CTRL and PLGA-I scaffolds. In fact, the ALP activity was significantly higher in the PLGA-I samples at 21 days compared to the CTRL samples, which is consistent with the gene expression results.

### CLAUSES

For reasons of completeness, various aspects of the invention are set out in the following numbered clauses:
Clause 1. A method for producing a synthetic bone graft comprising:
(a) Preparing an ink composition comprising α-TCP and one or more binders, this step comprising:
   a.1. preparing a binder solution comprising one or more non-water-soluble binders; or, alternatively, one or more water-soluble photo-crosslinkable binder(s), and
   a.2. adding α-TCP to the binder solution,
this step (a) further comprising, when the one or more binders are photo-crosslinkable monomer(s) or oligomer(s), the adding of one or more photoinitiator(s);
(b) 3D printing the synthetic bone graft; and
(c) Subjecting the synthetic bone graft to conditions providing cohesion to the bone graft, wherein this step comprises:
   c.1. reducing the solvent content of the bone graft; or, alternatively,
   c.2. crosslinking the one or more photo-crosslinkable monomer(s) or oligomer(s) in the presence of the photoinitiator.
Clause 2. The method of clause 1, which further includes the step (d) of hydrolysing the ceramic particles to give interlocked calcium deficient hydroxyapatite crystals.
Clause 3. The method of any one of the preceding clauses, wherein the binder solution comprises one or more non-water-soluble binders, particularly polyester binders.
Clause 4. The method of any one of the preceding clauses, wherein the one or more polyester binders are selected from the group consisting of polylactic acid (PLA), polyglycolic acid (PGA), copolymers of lactic acid and glycolic acid (i.e., polylactic-co-glycolic acid (PLGA)), polycaprolactone (PCL), and combinations thereof.
Clause 5. The method of any one of the preceding clauses, wherein the binder solution comprises PLA, PLLA, PGA, PLGA, PCL or a combination thereof.
Clause 6. The method of any one of the clauses 1-2, wherein the binder solution comprises one or more photo-crosslinkable binder(s), as well as a photoinitiator.
Clause 7. The method of clause 6, wherein the one or more photo-crosslinkable binders are selected from the acrylate binders, such as PEGDA and PEGDMA.
Clause 8. The method of any of the clauses 6-7, wherein the binder aqueous solution comprises, in addition to the photo-crosslinkable binder(s), one or more water-soluble binders other than those photo-crosslinkable.
Clause 9. The method of clause 8, wherein the other water-soluble binder(s) are poly(oxypropylene)-poly(oxyethylene) copolymers, such as poloxamers, polyethylene glycol (PEG).
Clause 10. The method of any of the clauses 8-9, wherein the one or more water-soluble photo-crosslinkable binder(s) are in a higher % w/w with respect to the % w/w of the other water-soluble binder(s).
Clause 11. The method of clause 10, wherein the one or more water-soluble photo-crosslinkable binder(s) are at a % w/w from 30 to 100% and the other water-soluble binder(s) are at a % w/w from to 40 wt %.
Clause 12. The method of any one of the preceding clauses, wherein the one or more binders are at % by weight, with respect to the total weight of the binder solution, from 5 to 80% w/w, particularly from 10 to 70% w/w.
Clause 13. The method of any one of the preceding clauses, wherein the weight ratio [binder solution:α-TCP] is from 0.1 to 2, particularly from 0.3 to 1.5, particularly the weight ratio is 0.2, 0.3, 0.4, 0.5, 0.6, 0.7, 0.8, 0.9, 1.0, 1.1, 1.2, 1.3, 1.4 or 1.5.
Clause 14. The method of any one of the preceding clauses, wherein step (a) further comprises adding a further hydroxyapatite compound in the range from 0.5 to 10% w/w with respect to the total weight of the ink composition, particularly from 1 to 5% w/w.
Clause 15. The method of any one of the preceding clauses 1-7, 12-14, wherein the binder(s) are non-water-soluble, particularly polyester(s), and the solvent is liquid at 25° C. and at 760 mmHg, and has a vapour pressure at 25° C. equal or greater than 15 mmHg.
Clause 16. The method of clause 15, wherein the solvent is selected among: methanol, ethanol, propanol, isopropanol, butanol, hexafluoroisopropanol (HFIP), carboxyl acids, sulfonic acids, formic acid, 1,4-Dioxane, tetrahydrofuran (THF), acetone, acetonitrile, dimethylformamide, dimethyl sulfoxide, include hexane, benzene, toluene, diethyl ether, chloroform, ethyl acetate, dichloromethane, methylene chloride, oxolane, and any combination thereof; particularly the solvent is selected from 1,4-dioxane, dichloromethane, pyridine, chloroform, methylene chloride and, hexafluoroisopropanol (HFIP).
Clause 17. The method of any one of the preceding clauses, wherein the ink composition prepared in step (a) is one selected from those provided in Tables 1 to 3.
Clause 18. The method of any one of the preceding clauses, wherein the ink composition comprises a polyester binder solution and step (c) is performed by evaporating of the solvent, rising/washing with/in water or dissolution/dilution in water or sublimation by freeze-drying; particularly evaporating the solvent and sublimation by freeze-drying.
Clause 19. The method of any one of the preceding clauses, wherein step (c) is performed by crosslinking acrylate binder(s) in the presence of one or more photoinitiator(s).
Clause 20. The method of any one of the preceding clause, wherein step (c) is performed with PEGDA or PEGDMA and the photoinitiator is selected from benzoylphosphine oxides, such as 2,4,6-trimethylbenzoyldiphenylphosphine oxide (Lucirin TPO), Phenyl-bis-(2,4,6-trimethylbenzoyl)-phosphinoxide (BAPO).
Clause 21. The method of any one of the preceding clauses, wherein step (d) comprises contacting the bone graft with an aqueous solution.
Clause 22. The method of clause 21, wherein step (d) comprises the immersion in water or in an aqueous solution comprising carbonate, bicarbonate, silicate, Ca, Mg, Sr, Ce, Al, Zn, Ag, Co, Cu, other transition metals or any combination thereof.
Clause 23. The method of clause 22, wherein step (d) comprises the immersion of the bone graft in water and heating at a temperature equal or above 90° C., particularly equal or above 100° C. and a pressure from 0.5 to 4 atm., of absolute pressure.
Clause 24. The method of clause 23, wherein step (d) comprises the immersion of the bone graft in water and autoclaving at a temperature of 100° C. and a pressure from 0.5 to 2 atm, particularly from 0.5 to 1.5 atm, particularly at 1 atm., of absolute pressure.
Clause 25. The method of any one of the clauses 2-24, wherein step (d) is performed for a period of time of 120 minutes or less, particularly from 40 to 80 minutes, particularly from 45 to 65 minutes, particularly for 55 min.
Clause 26. The method of any one of the clauses 2-25, wherein step (d) comprises immersing the bone graft in water at a temperature from 100 to 110° C., for 45 to 65 minutes, at 0.5 to 1.5 atm of absolute pressure.
Clause 27. The method of any one of the clauses 2-26, wherein step (d) comprises immersing the bone graft in water at a temperature of 100, for 55 minutes, at 1 atm, absolute pressure.
Clause 28. The method of any one of the preceding clauses 2-21, wherein step (d) comprises grafting the synthetic bone graft resulting from step (c) in an animal body.
Clause 29. The method of any one of the preceding clauses 2-20, wherein step (d) comprises subjecting the bone graft resulting from step (c) to water vapor atmosphere.
Clause 30. The method of any one of the preceding clauses, which comprises the coating of the scaffold resulting from step (d) with a binder solution as defined in any of the preceding claims.
Clause 31. The method of any one of the preceding clauses, comprising:
   (a) preparing an ink composition comprising 10-60 wt % of PLGA dissolved in 1,4-dioxane, and α-TCP at a weight ratio binder solution:α-TCP from 0.4 to 0.7;
   (b) 3D-printing;
   (c) evaporating the 1,4-dioxane; and
   (d) immersing the scaffold in water at 100° C., 1 atm, for less than 60 min, particularly at 55 min.
Clause 32. A 3D-printed bone graft made of a composition comprising a ceramic matrix which is in admixture with a binder matrix, wherein:
   the ceramic matrix comprises a crystalline phase including interlocked calcium-deficient hydroxyapatite (CDHA) crystals; and
   the binder matrix is made from one or more non-water-soluble binders; or, alternatively, one or more water-soluble photo-crosslinkable binders;
   the ceramic matrix is at a weight percentage of at least 50 wt % with respect to the total weight of the composition, and
   the binder matrix is at a weight percentage in the range from 5 to 40 wt % with respect to the total weight of the composition.
Clause 33. The 3D-printed bone graft of clause 32, wherein the CDHA crystals are at a percentage by weight from 30 to 100%, particularly from 40 to 75%, or particularly from 60 to 70% with respect to the total weight of the crystalline phase of the ceramic matrix.
Clause 34. The 3D-printed bone graft of any of the clauses 32-33, wherein the ceramic matrix further comprises α-TCP and/or β-TCP.
Clause 35. The 3D-printed bone graft of clause 34, wherein the crystalline phase included in the ceramic matrix comprises α-TCP at a weight percentage from 5 to 25%, particularly from 10 to 20%, with respect to the total weight of the crystalline phase of the ceramic matrix.
Clause 36. The 3D-printed bone graft of any one of the clauses 32-35, wherein the crystalline phase included in the ceramic matrix comprises β-TCP at a weight percentage from 15 to 30%, particularly from 18 to 25%, with respect to the total weight of the crystalline phase of the ceramic matrix.
Clause 37. The 3D-printed bone graft of any one of the preceding clauses, wherein the ceramic matrix comprises a crystalline phase with the following composition:

$$β - TCP : 20 - 20.2 \% ;$$

$$α - TCP : 12.4 - 1 5.3 \%$$

$$CDHA : 64.4 - 67.5 \%$$

wherein the percentages by weight are determined with respect to the total weight of the crystalline phase of the ceramic matrix, and the sum of the components provides 100%.
Clause 38. The 3D-printed bone graft of any one of the preceding clauses, wherein the CDHA crystals are doped with one or more groups selected from the group consisting of carbonate, bicarbonate, silicate, Ca, Mg, Sr, Ce, Al, Zn, Ag, Co, Cu and other transition metals.
Clause 39. The 3D-printed bone graft as defined in any one of the clauses 32-38, which is obtainable by a method as defined in any one of the clauses 1-31.
Clause 40. A 3D-printed bone graft made of a composition comprising α-TCP particles in admixture with a binder matrix, the polymeric matrix being made from one or more non-water-soluble binders; or, alternatively, from one or more water-soluble photo-crosslinked binders;
wherein the α-TCP particles are at a weight percentage of least 50 wt % with respect to the total weight of the composition, and the polymeric matrix is at a weight percentage in the range from 5 to 40 wt % with respect to the total weight of the composition.
Clause 41. The 3D-printed bone graft of clause 39, which is obtainable by the method as defined in any of the clauses 1-265.
Clause 42. The 3D-printed bone graft of any one of the preceding clauses, wherein the polymeric matrix is made from one or more polyester binders.
Clause 43. The 3D-printed bone graft of any one of the preceding clauses, wherein the one or more polyester binders are selected from PLA, PLLA, PGA, PLGA, PCL and any combination thereof.
Clause 44. The 3D-printed bone graft of any one of the preceding clauses, wherein the binder matrix is made from one or more photocrosslinked binder(s), and further includes a photoinitiator.
Clause 45. The 3D-printed bone graft of the preceding clause, wherein the one or more photocrosslinked binder(s) are acrylates, particularly PEGDA and PEGDMA.
Clause 46. The 3D-printed bone graft of any one of the preceding clauses, wherein the photoinitiator is selected from benzoylphosphine oxides, such as 2,4,6-trimethylbenzoyldiphenylphosphine oxide (Lucirin TPO), Phenyl-bis-(2,4,6-trimethylbenzoyl)-phosphinoxide (BAPO).
Clause 47. The 3D-printed bone graft of any one of the preceding clauses, wherein the polymeric matrix is at a weight % from 5 to 30% with respect to the total weight of the composition, particularly from 7 to 30% w/w.
Clause 48. The 3D-printed bone graft of any one of the preceding clauses, which has macro-porosity in the range of 10% to 80%, from 30% to 70%, or from 40% to 60%, as determined by calculating the difference between the total porosity and the nano-micro porosity, wherein the nano-micro porosity is determined by mercury intrusion porosimetry.
Clause 49. The 3D-printed bone graft of any one of the preceding claims, which has nano-micro porosity in the range of 0.1% to 30%, particularly from 0.1 to 15%.
Clause 50. The 3D-printed bone graft of any one of the preceding claims, which has an apparent density below 2 g/cm, particularly below 1.5 g/cm, as determined by the quotient of the scaffold mass over the scaffold equivalent cubic volume (2×1×1 cm³) obtained from the measurements of the scaffold length, width and height.
Clause 51. The 3D-printed bone of any one of the preceding claims, which has a specific surface area (SSA) in the range of 1 to 15 m2/g, as determined by nitrogen adsorption and BET analysis.

1. A method for producing a synthetic bone graft comprising:
(a) Preparing an ink composition comprising α-TCP and one or more binders, this step comprising:
a.1. preparing a binder solution comprising one or more non-water-soluble binders or one or more water-soluble photo-crosslinkable binder(s) in a solvent, and
a.2. adding α-TCP to the binder solution,
this step (a) further comprising, when the one or more binders are photo-crosslinkable binder(s), the adding of one or more photoinitiator(s);
(b) 3D printing the synthetic bone graft; and
(c) Subjecting the synthetic bone graft to conditions providing cohesion to the bone graft, wherein this step comprises:
c.1. reducing the solvent content of the bone graft; or
c.2. crosslinking the one or more photo-crosslinkable binder(s) in the presence of the photoinitiator(s); and
wherein:
the term “non-water-soluble” refers to a binder having a solubility in water at 25° C. lower than 10 mg per mL; and the term “water-soluble” refers to a photo-crosslinkable binder having a solubility in water at 25° C. equal or higher than 10 mg per mL.

2. The method of claim 1, which further includes a step (d) comprising the hydrolysis of the α-TCP to give interlocked calcium deficient hydroxyapatite crystals.

3. The method of claim 1, wherein the binder solution comprises one or more non-water-soluble binders including one or more polyester binders.

4. The method of claim 3, wherein the one or more polyester binders are selected from the group consisting of polylactic acid (PLA), polyglycolic acid (PGA), copolymers of lactic acid and glycolic acid, polycaprolactone (PCL), and combinations thereof.

5. The method of claim 1, wherein the binder solution comprises the one or more photo-crosslinkable binder(s) and includes one or more photo-crosslinkable acrylate binders, as well as a photoinitiator.

6. The method of claim 1, wherein the one or more binders are at % by weight, with respect to the total weight of the binder solution, and the one or more binders are from 5 to 80% w/w.

7. The method of claim 1, wherein a weight ratio of binder solution to α-TCP particles is from 0.1 to 2.

8. The method of claim 1, wherein the one or more binder(s) are non-water-soluble and include polyester(s), and the solvent is liquid at 25° C. and at 760 mmHg, and has a vapour pressure at 25° C. equal or greater than 15 mmHg.

9. The method of claim 1, wherein the ink composition prepared in step (a) is one selected from compositions 1-3:
1. (i) 30-100 wt. % Poly(ethylene glycol) diacrylate (PEGDA), Poly(ethylene glycol) dimethacrylate (PEGDMA),
(ii) 0.1-40 wt. % Poloxamer (Kolliphor 407),
(iii) 0-10 μl Phenylbis(2,4,6-trimethylbenzoyl)phosphine oxide) (BAPO),
(iv) water, and
(v) α-TCP particles in an amount sufficient to produce a liquid to powder ratio in the ink composition of 0.20-0.55:
2. (i) 6-15 wt. % Poly(L-lactic acid) (PLLA),
(ii) 3-15 wt. % Poloxamer (Kolliphor 407)
(iii) Dichloromethane (DCM),
(iv) α-TCP particles in an amount sufficient to produce a liquid to powder ratio in the ink composition of 0.8-1.3;
3. (i) 15-50 wt. % Poly(L-lactic acid) (PLLA) or Poly(lactic co-glycolic acid) (PLGA),
(ii) 1,4-Dioxane,
(iii) α-TCP particles in an amount sufficient to produce a liquid to powder ratio in the ink composition of 0.4-1.0.

10. The method of claim 1, wherein the ink composition comprises a polyester binder solution and step (c) is performed by evaporating the solvent, rising/washing with/in water, dissolution/dilution in water, or sublimation by freeze-drying.

11. The method of claim 1, wherein step (c) is performed by crosslinking acrylate binder(s) in the presence of the one or more photoinitiator(s).

12. The method of claim 2, wherein step (d) comprises:
contacting the bone graft with an aqueous solution; or
grafting the synthetic bone graft resulting from step (c) in an animal body; or
subjecting the bone graft resulting from step (c) to water vapor atmosphere.

13. A 3D-printed bone graft made of a composition comprising a ceramic matrix which is in admixture with a binder matrix, wherein:
the ceramic matrix comprises a crystalline phase including interlocked calcium-deficient hydroxyapatite (CDHA) crystals;-
the binder matrix is made from one or more non-water-soluble binders; or the binder matrix is made from one or more water-soluble photo-crosslinkable binders;
the ceramic matrix is at a weight percentage of at least 50 wt % with respect to the total weight of the composition, and
the binder matrix is at a weight percentage in the range from 5 to 40 wt % with respect to the total weight of the composition.

14. The 3D-printed bone graft of claim 13, which comprises one or more of the following features selected from the group consisting of.
(a) the CDHA crystals are at a percentage by weight from 30 to 100%, with respect to the total weight of the crystalline phase of the ceramic matrix;
(b) the ceramic matrix further comprises α-TCP and/or β-TCP;
(c) the crystalline phase included in the ceramic matrix comprises α-TCP at a weight percentage from 5 to 25% with respect to the total weight of the crystalline phase of the ceramic matrix;
(d) the crystalline phase included in the ceramic matrix comprises j-TCP at a weight percentage from 15 to 30% with respect to the total weight of the crystalline phase of the ceramic matrix; and
(e) the ceramic matrix comprises a crystalline phase with the following composition:
$$β - TCP : 20 - 20.2 \% ;$$
$$α - TCP : 12.4 - 1 5.3 \%$$
$$CDHA : 64.4 - 67.5 \%$$
wherein the percentages by weight are determined with respect to the total weight of the crystalline phase of the ceramic matrix, and the sum of the components provides 100%.

15. A 3D-printed bone graft made of a composition comprising:
α-TCP particles in admixture with a binder matrix, the binder matrix being made from one or more non-water-soluble binders, or from one or more water-soluble photo-crosslinked binders; and
wherein the α-TCP particles are at a weight percentage of least 50 wt % with respect to the total weight of the composition, and the binder matrix is at a weight percentage in the range from 5 to 40 wt % with respect to the total weight of the composition.

16. The 3D-printed bone graft of claim 15, which is obtainable by the method as defined in claim 1.

17. The 3D-printed bone graft of claim 15, which further comprises one or more of the technical features selected from the group consisting of:
(a) the binder matrix is made from one or more polyester binders;
(b) alternatively to (a), the binder matrix is made from one or more photo-crosslinked binder(s), and further includes one or more photoinitiator(s);
(c) the binder matrix, as defined in (a) or (b), is at a weight % from 5 to 30% with respect to the total weight of the composition;
(d) the 3D-printed bone graft has macro-porosity in the range of 10% to 80% as determined by calculating the difference between the total porosity and the nano-micro porosity, wherein the nano-micro porosity is determined by mercury intrusion porosimetry;
(e) the 3D-printed bone graft nano-micro porosity in the range of 0.1% to 30%;
(f) the 3D-printed bone graft has an apparent density below 2 g/cm as determined by the quotient of the scaffold mass over the scaffold equivalent cubic volume (2×1×1 cm3) obtained from the measurements of the scaffold length, width and height; and
(g) the 3D-printed bone graft has a specific surface area (SSA) in the range of 1 to 15 m2/g, as determined by nitrogen adsorption and BET analysis.

18. The method of claim 5, wherein the one or more photo-crosslinkable acrylate binders is at least one of PEGDA and PEGDMA.

19. The method of claim 1, wherein the one or more binders are at % by weight, with respect to the total weight of the binder solution, and the one or more binders are from 10 to 70% w/w.

20. The method of claim 1, wherein a weight ratio of binder solution to α-TCP particles is from 0.3 to 1.5.
